(19) United States
(12) Patent Application Publication
(10) Pub. No.: US 2025/0109437 A1
(43) Pub. Date: Apr. 3, 2025

(54) KITS AND METHODS FOR BIOLOGICAL SEQUENCING OPERATIONS

(71) Applicant: ILLUMINA, INC., San Diego, CA (US)

(72) Inventors: Jolene Lei, San Diego, CA (US); Jie Mao, San Diego, CA (US); Daniel Solis, Escondido, CA (US); Jelena Stojakovic, San Diego, CA (US)

(21) Appl. No.: 18/895,767

(22) Filed: Sep. 25, 2024

Related U.S. Application Data
(60) Provisional application No. 63/586,913, filed on Sep. 29, 2023.

(51) Int. Cl. C12Q 1/6874; B01L 3/00

(52) U.S. Cl. CPC C12Q 1/6874; B01L 3/502761; B01L 2200/0668; B01L 2300/0636; B01L 2300/0883

(57) ABSTRACT

A kit for biological sequencing operations includes a flow cell including a plurality of depressions having a first diameter. The kit further includes a suspension including: a liquid carrier; and a plurality of functionalized nanostructures dispersed throughout the liquid carrier. Each of the plurality of functionalized nanostructures has a second diameter that is equal to or less than the first diameter. Each of the plurality of functionalized nanostructures includes: a nanostructure core; and a plurality of primers attached to the nanostructure core. The kit further includes a plurality of mechanical loading beads having a third diameter that is greater than the first diameter.

### CROSS-REFERENCE TO RELATED APPLICATION

This application claims the benefit of U.S. Provisional Application Ser. No. 63/586,913, filed Sep. 29, 2023, the contents of which is incorporated by reference herein in its entirety.

### REFERENCE TO SEQUENCE LISTING

The instant application contains a Sequence Listing which has been submitted electronically in XML file format and is hereby incorporated by reference in its entirety. Said XML copy, created on Sep. 15, 2024 is named ILI263B_IP-2647-US_Sequence_Listing.xml and is 17,741 bytes in size.

### BACKGROUND

Various protocols in biological or chemical research involve performing a large number of controlled reactions on local support surfaces or within predefined reaction chambers, or reactive areas. The reactions may then be observed or detected, and subsequent analysis may help identify or reveal properties of chemicals involved in the controlled reactions. In some examples, the reactions generate fluorescence, and thus an optical system that is configured for fluorescence detection may be used to analyze the controlled reactions. In other examples, the controlled reactions alter charge, conductivity, or some other electrical property, and thus an electronic system may be used for detection.

### SUMMARY

Functionalized nanostructures and kits and methods utilizing the same are disclosed herein. The functionalized nanostructures disclosed herein include a surface chemistry that is suitable for seeding and clustering library templates. The functionalized nanostructures may be loaded into depressions that are defined in a substrate surface. The functionalized nanostructures disclosed herein enable pre-clustered nanostructures, including amplicons of the library templates, to be formed on the nanostructures prior to the introduction of the functionalized nanostructures into the depressions (and thus the nanostructures may be used for off-flow cell (i.e., off-board) preparation). Alternatively, non-pre-clustered functionalized nanostructures can be used to generate amplicons of library templates after the nanostructures have been introduced into the depressions (and thus the nanostructures may be used for on-flow cell (i.e., on-board) library preparation).

After the functionalized nanostructures are introduced into the flow cell, a plurality of mechanical loading beads disclosed herein may be used to physically displace the functionalized nanostructures, such that the functionalized nanostructures become disposed within (e.g., forced into) the depressions that are defined in the flow cell surface. Use of the plurality of mechanical loading beads disclosed herein results in a higher number of occupied depressions (i.e., those containing at least one functionalized nanostructure) relative to the number of depressions that are occupied when the mechanical loading beads are not used. When a higher number of available depressions are occupied by functionalized nanostructures, more amplicons are present across the flow cell surface and are subjected to analysis, thus increasing the amount of data that is generated.

Overall, the functionalized nanostructures and mechanical loading beads and their uses in the flow cell disclosed herein aid in improving sequencing metrics.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A is a schematic illustration of an example of a non-pre-clustered functionalized nanostructure;

FIG. 1B is a schematic illustration of an example of a pre-clustered functionalized nanostructure;

FIG. 2A is a top view of an example of a flow cell;

FIG. 2B is an enlarged, and partially cutaway view of one example of an architecture in a flow channel of the flow cell;

FIG. 3A through FIG. 3C are schematic views that together illustrate one example of a method disclosed herein, where FIG. 3A depicts the introduction of functionalized nanostructures into a flow cell including depressions, FIG. 3B depicts the introduction of a plurality of mechanical loading beads into the structure of FIG. 3A and the displacement of the functionalized nanostructures into the depressions, and FIG. 3C depicts the removal of the plurality of mechanical loading beads from the structure of FIG. 3B;

FIG. 4 depicts an image obtained from a scanning electron microscope (SEM) that shows clustered functionalized nanostructures within depressions of a flow cell after a sequencing operation has been performed therewith;

FIG. 5A is a graph depicting intensity measurements (Y axis, arbitrary units) versus the number of sequencing cycles (X axis, Cycle #) obtained from an example of a flow cell disclosed herein that was loaded with a plurality of the functionalized nanostructures disclosed herein;

FIG. 5B is a graph depicting intensity measurements (Y axis, arbitrary units) versus the number of sequencing cycles (X axis, Cycle #) obtained from a comparative flow cell that was free of functionalized nanostructures; and

FIG. 6 is a schematic illustration of a complementary metal-oxide semiconductor imaging device that is coupled to a substrate.

### DETAILED DESCRIPTION

Functionalized nanostructures are disclosed herein. These nanostructures may be included in kits and used in methods that lead to improved sequencing metrics.

### Definitions

It is to be understood that terms used herein will take on their ordinary meaning in the relevant art unless specified otherwise. Several terms used herein and their meanings are set forth below.

The singular forms “a”, “an”, and “the” include plural referents unless the context clearly dictates otherwise.

The terms comprising, including, containing and various forms of these terms are synonymous with each other and are meant to be equally broad.

The terms top, bottom, lower, upper, adjacent, on, etc. are used herein to describe the flow cell and/or the various components of the flow cell. It is to be understood that these directional terms are not meant to imply a specific orientation, but are used to designate relative orientation between components. The use of directional terms should not be interpreted to limit the examples disclosed herein to any specific orientation(s).

The terms first, second, etc. also are not meant to imply a specific orientation or order, but rather are used to distinguish one component from another.

An “acrylamide monomer” is a monomer with the structure

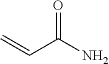

or a monomer including an acrylamide group. Examples of the monomer including an acrylamide group include azido acetamido pentyl acrylamide

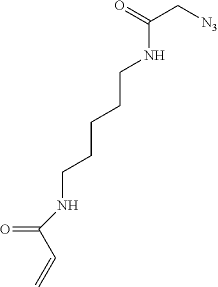 [SMILES: C=CC(=O)NCCCCCNC(=O)CN=[N+]=[N-]]

### and N-isopropylacrylamide:

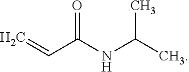

Other acrylamide monomers may be used.

An “aldehyde,” as used herein, is an organic compound containing a functional group with the structure-CHO, which includes a carbonyl center (i.e., a carbon double-bonded to oxygen) with the carbon atom also bonded to hydrogen and an R group, such as an alkyl or other side chain. The general structure of an aldehyde is:

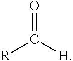

As used herein, “alkyl” refers to a straight or branched hydrocarbon chain that is fully saturated (i.e., contains no double or triple bonds). The alkyl group may have 1 to 20 carbon atoms. Example alkyl groups include methyl, ethyl, propyl, isopropyl, butyl, isobutyl, tertiary butyl, pentyl, hexyl, and the like. As an example, the designation “C1-4 alkyl” indicates that there are one to four carbon atoms in the alkyl chain, i.e., the alkyl chain is selected from the group consisting of methyl, ethyl, propyl, iso-propyl, n-butyl, isobutyl, sec-butyl, and t-butyl.

As used herein, “alkenyl” refers to a straight or branched hydrocarbon chain containing one or more double bonds. The alkenyl group may have 2 to 20 carbon atoms. Example alkenyl groups include ethenyl, propenyl, butenyl, pentenyl, hexenyl, and the like.

As used herein, “alkyne” or “alkynyl” refers to a straight or branched hydrocarbon chain containing one or more triple bonds. The alkynyl group may have 2 to 20 carbon atoms.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms. Examples of aryl groups include phenyl, naphthyl, azulenyl, and anthracenyl.

An “amino” functional group refers to an —NRₐR_{b} group, where Rₐ and R_{b} are each independently selected from hydrogen (e.g.,

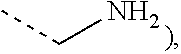

C1-6 alkyl, C2-6 alkenyl, C2-6 alkynyl, C3-7 carbocycle, C6-10 aryl, 5-10 membered heteroaryl, and 5-10 membered heterocyclyl, as defined herein.

As used herein, the terms “attached” refers to the state of two things being joined, fastened, adhered, connected or bound to each other, either indirectly or directly. As an example of an indirect attachment, a primer is attached to a nanostructure core via an intervening hydrogel coating that is applied on the nanostructure core (and thus the primer is “indirectly attached” to the nanostructure core). As an example of direct attachment, a primer can be bonded to a hydrogel by a covalent or non-covalent bond (and thus the primer is “directly attached” to the hydrogel). A covalent bond is characterized by the sharing of pairs of electrons between atoms. A non-covalent bond is a physical bond that does not involve the sharing of pairs of electrons and can include, for example, hydrogen bonds, ionic bonds, van der Waals forces, hydrophilic interactions and hydrophobic interactions. Other examples of attachment include magnetic attachment or electrostatic attachment.

An “azide” or “azido” functional group refers to —N₃.

As used herein, “carbocycle” means a non-aromatic cyclic ring or ring system containing only carbon atoms in the ring system backbone. When the carbocycle is a ring system, two or more rings may be joined together in a fused, bridged or spiro-connected fashion. Carbocycles may have any degree of saturation, provided that at least one ring in a ring system is not aromatic. Thus, carbocycles include cycloalkyls, cycloalkenyls, and cycloalkynyls. The carbocycle group may have 3 to 20 carbon atoms. Examples of carbocycle rings include cyclopropyl, cyclobutyl, cyclopentyl, cyclohexyl, cyclohexenyl, 2,3-dihydro-indene, bicyclo[2.2.2]octanyl, adamantyl, and spiro[4.4]nonanyl.

As used herein, the term “carboxylic acid” or “carboxyl” refers to —COOH.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s). In some examples, cycloalkyl groups can contain 3 to 8 atoms in the ring(s). A cycloalkyl group may be unsubstituted or substituted. Example cycloalkyl groups include cyclopropyl, cyclobutyl, cyclopentyl, cyclohexyl, cycloheptyl, and cyclooctyl.

As used herein, “cycloalkenyl” or “cycloalkene” means a carbocycle ring or ring system having at least one double bond, wherein no ring in the ring system is aromatic. Examples include cyclohexenyl or cyclohexene and norbornenyl or norbornene. Also as used herein, “heterocycloalkenyl” or “heterocycloalkene” means a carbocycle ring or ring system with at least one heteroatom in ring backbone, having at least one double bond, wherein no ring in the ring system is aromatic.

As used herein, “cycloalkynyl” or “cycloalkyne” means a carbocycle ring or ring system having at least one triple bond, wherein no ring in the ring system is aromatic. An example is cyclooctyne. Another example is bicyclononyne. Also as used herein, “heterocycloalkynyl” or “heterocycloalkyne” means a carbocycle ring or ring system with at least one heteroatom in ring backbone, having at least one triple bond, wherein no ring in the ring system is aromatic.

The term “depositing,” as used herein, refers to any suitable application technique, which may be manual or automated, and, in some instances, results in modification of the surface properties. Generally, depositing may be performed using vapor deposition techniques, coating techniques, grafting techniques, or the like. Some specific examples include chemical vapor deposition (CVD), spray coating (e.g., ultrasonic spray coating), spin coating, dunk or dip coating, doctor blade coating, puddle dispensing, flow through coating/deposition, aerosol printing, screen printing, microcontact printing, inkjet printing, or the like.

The term “each,” when used in reference to a collection of items, is intended to identify an individual item in the collection, but does not necessarily refer to every item in the collection. Exceptions can occur if explicit disclosure or context clearly dictates otherwise.

The term “epoxy” (also referred to as a glycidyl or oxirane group) as used herein refers to

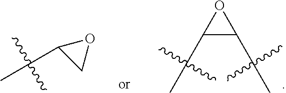

As used herein, the term “flow cell” is intended to mean a vessel having a flow channel where a reaction can be carried out, an inlet for delivering reagent(s) to the flow channel, and an outlet for removing reagent(s) from the flow channel. In some examples, the flow cell accommodates the detection of the reaction that occurs in the flow cell. For example, the flow cell can include one or more transparent surfaces allowing for the optical detection of arrays, optically labeled molecules, or the like.

As used herein, a “flow channel” or “channel” may be (i) an area defined between two bonded components or may be (ii) a concave area, or lane, defined in a single substrate. In either case, the “flow channel” or “channel” can selectively receive a liquid sample, reagents, etc. In some examples, the flow channel may be defined between two substrates, and thus the flow channel may be in fluid communication with functionalized nanostructures disposed within depressions on either of the two substrates. In other examples, the flow channel may be defined between one substrate and a lid, and thus the flow channel may be in fluid communication with the functionalized nanostructures within depressions of the one substrate. In still other examples, the flow channel may be defined by a concave area that is formed in a substrate surface, and thus the flow channel may be in fluid communication with functionalized nanostructures within depressions of the concave area.

In some instances, the term “functionalized nanostructure” or “nanostructure” refers to i) a nanostructure core, ii) a polymeric hydrogel attached to the nanostructure core, and iii) a plurality of primers attached to side chains or arms of the polymeric hydrogel. In other instances, the terms refer to a polymeric hydrogel core with primers attached thereto. In each of these instances, when the functionalized nanostructure has not yet been exposed to seeding and amplification, it may be referred to herein as being “non-pre-clustered.” Non-pre-clustered functionalized nanostructures can be introduced into the depressions of the flow cell and then exposed to seeding and amplification. In other instances, the functionalized nanostructure may be referred to as being “pre-clustered,” meaning that each of the plurality of primers is seeded with a strand of template DNA (i.e., a library template) and exposed to amplification (i.e., generates amplicons) prior to the introduction of the nanostructures into the depressions of a flow cell.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members.

As used herein, “heterocycle” means a non-aromatic cyclic ring or ring system containing at least one heteroatom in the ring backbone. Heterocycles may be joined together in a fused, bridged or spiro-connected fashion. Heterocycles may have any degree of saturation provided that at least one ring in the ring system is not aromatic. In the ring system, the heteroatom(s) may be present in either a non-aromatic or aromatic ring. The heterocycle group may have 3 to 20 ring members (i.e., the number of atoms making up the ring backbone, including carbon atoms and heteroatoms). In some examples, the heteroatom(s) are O, N, or S.

The term “hydrazine” or “hydrazinyl” as used herein refers to a-NHNH₂ group.

As used herein, the term “hydrazone” or “hydrazonyl” refers to a

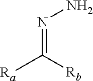

group in which Rₐ and R_{b} are each independently selected from hydrogen, C1-6 alkyl, C2-6 alkenyl, C2-6 alkynyl, C3-7 carbocycle, C6-10 aryl, 5-10 membered heteroaryl, and 5-10 membered heterocyclyl, as defined herein.

As used herein, “hydroxy” or “hydroxyl” refers to an —OH group.

The term “hydrogel” or “polymeric hydrogel” refers to a semi-rigid polymer that is permeable to liquids and gases. The hydrogel can swell when liquid (e.g., water) is taken up and can contract when liquid is removed, e.g., by drying. While a hydrogel may absorb water, it is not water-soluble.

As used herein, the term “interstitial region” refers to an area, e.g., of a substrate, that separates flow cell depressions from one another. The separation provided by an interstitial region can be partial or full separation.

As used herein, a “nanostructure core” or “core” refers to a central material included in a functionalized nanostructure. In some instances, the core is coated with another material that is capable of attaching primers thereto. In other instances, the core is comprised of a material that is capable of attaching primers thereto.

“Nitrile oxide,” as used herein, means a “RₐC≡N⁺O⁻” group in which Rₐ is defined herein. Examples of preparing nitrile oxide include in situ generation from aldoximes by treatment with chloramide-T or through action of base on imidoyl chlorides [RC(Cl)=NOH] or from the reaction between hydroxylamine and an aldehyde.

“Nitrone,” as used herein, means a

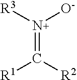

group in which R¹, R², and R³ may be any of the Rₐ and R_{b} groups defined herein, except that R³ is not hydrogen (H).

As used herein, a “nucleotide” includes a nitrogen containing heterocyclic base, a sugar, and one or more phosphate groups. Nucleotides are monomeric units of a nucleic acid sequence. In RNA, the sugar is a ribose, and in DNA, the sugar is a deoxyribose, i.e. a sugar lacking a hydroxyl group that is present at the 2′ position in ribose. The nitrogen containing heterocyclic base (i.e., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. A nucleic acid analog may have any of the phosphate backbone, the sugar, or the nucleobase altered. Examples of nucleic acid analogs include, for example, universal bases or phosphate-sugar backbone analogs, such as peptide nucleic acid (PNA). A “labeled nucleotide” is a nucleotide that has at least an optical label attached thereto. Examples of optical labels include any dye that is capable of emitting an optical signal in response to an excitation wavelength.

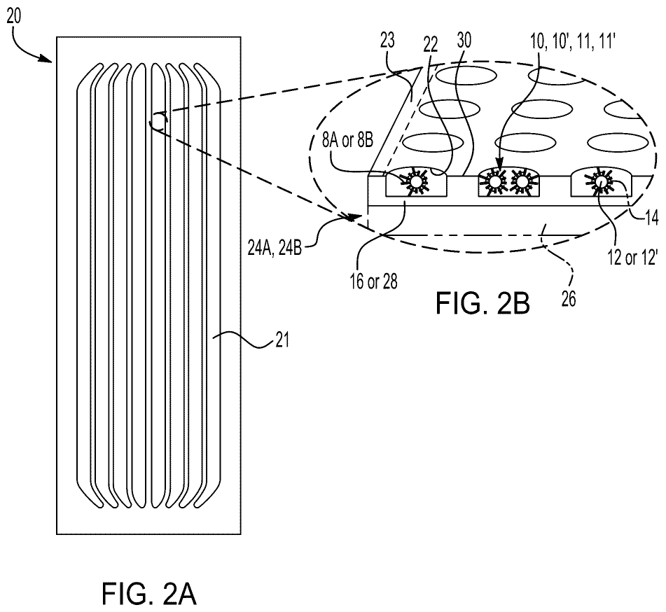

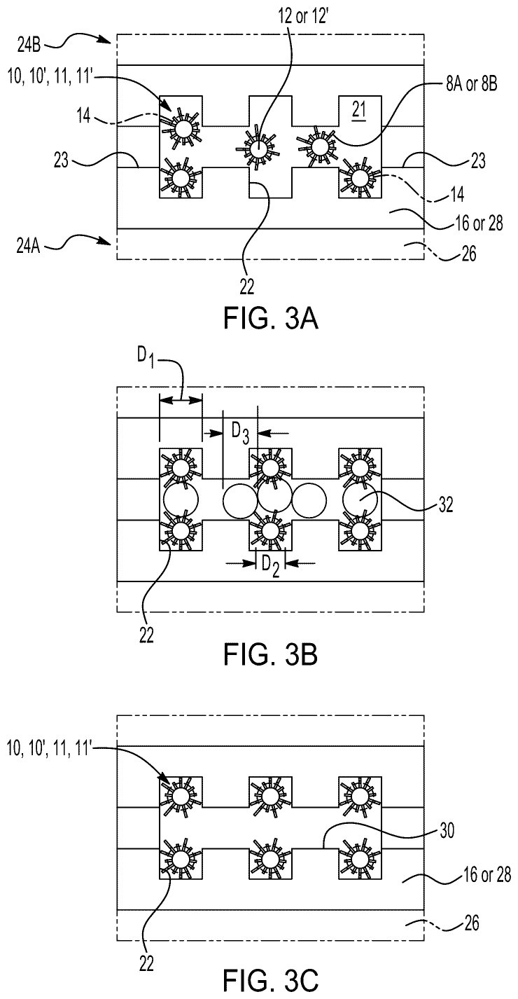

In some examples, the term “over” may mean that one component or material is positioned directly on another component or material. When one is directly on another, the two are in contact with each other. In FIG. 3C, for example, the functionalized nanostructures 10, 10′, 11, 11′ are shown as being within the depressions 22, such that the nanostructures 10, 10′, 11, 11′ are “over” the substrate 16 or the layer 28 (e.g., there is no intervening material between the nanostructures 10, 10′, 11, 11′ within the depressions 22 and the substrate 16 or the layer 28). In other examples, the term “over” may mean that one component or material is positioned indirectly on another component or material. By indirectly on, it is meant that a gap or an additional component or material may be positioned between the two components or materials. In FIG. 2B, for example, when both a substrate 26 and a layer 28 are utilized and the depressions 22 are defined in the layer 28, the nanostructures 10, 10′, 11, 11′ are indirectly on the substrate 26. The layer 28 is positioned therebetween.

A “patterned resin” refers to any material that can have depressions defined therein. Specific examples of resins and techniques for patterning the resins will be described further below.

As used herein, the term “primer” is defined as a single stranded nucleic acid sequence (e.g., single stranded DNA). Some primers, referred to herein as amplification primers, serve as a starting point for template amplification and cluster generation. Other primers, referred to herein as sequencing primers, serve as a starting point for DNA synthesis. The 5′ terminus of the primer may be modified to allow a coupling reaction with a functional group of the core or coating overlying the core. The primer length can be any number of bases long and can include a variety of non-natural nucleotides. In an example, the sequencing primer is a short strand, ranging from 10 to 60 bases, or from 20 to 40 bases.

The term “substrate” refers to a support material that can be patterned with depressions, or that can include an additional layer thereon that can be patterned with depressions (e.g., using nanoimprint lithography). For example, the term may refer to a single layer substrate (such as the substrate 16 depicted in FIG. 2B), or a substrate 26 having a layer 28 thereon that forms a multi-layered structure (as further depicted by the dashed lines in FIG. 2B).

“Surface chemistry,” as defined herein, refers to primers that are present on functionalized nanostructures.

The term “tantalum pentoxide” refers to the inorganic compound with the formula Ta₂O₅. This compound is transparent, having a transmittance ranging from about 0.25 (25%) to 1 (100%), to wavelengths ranging from about 0.35 μm (350 nm) to at least 1.8 μm (1800 nm). A “tantalum pentoxide substrate” may comprise, consist essentially of, or consist of Ta₂O₅. In examples where it is desirable for the tantalum pentoxide substrate to transmit electromagnetic energy having any of these wavelengths, the substrate may consist of Ta₂O₅ or may comprise or consist essentially of Ta₂O₅ and other components that will not interfere with the desired transmittance of the substrate.

A “thiol” functional group refers to —SH.

As used herein, the terms “tetrazine” and “tetrazinyl” refer to six-membered heteroaryl group comprising four nitrogen atoms. Tetrazine can be optionally substituted.

“Tetrazole,” as used herein, refer to five-membered heterocyclic group including four nitrogen atoms. Tetrazole can be optionally substituted.

The term “transparent” refers to a material, e.g., in the form of a substrate or layer, that is transparent to a particular wavelength or range of wavelengths. Transparency may be quantified using transmittance, i.e., the ratio of light energy falling on a body to that transmitted through the body. The transmittance of a transparent substrate or a transparent layer will depend upon the thickness of the substrate or layer and the wavelength of light. In the examples disclosed herein, the transmittance of the transparent substrate or the transparent layer may range from 0.25 (25%) to 1 (100%). The material of the substrate or layer may be a pure material, a material with some impurities, or a mixture of materials, as long as the resulting substrate or layer is capable of the desired transmittance. Additionally, depending upon the transmittance of the substrate or layer, the time for light exposure and/or the output power of the light source may be increased or decreased to deliver a suitable dose of light energy through the transparent substrate and/or layer to achieve the desired effect (e.g., introducing excitation wavelengths to a substrate surface).

### Kit for Biological Sequencing Operations

Disclosed herein is a kit for biological sequencing operations comprising a flow cell including (i) a plurality of depressions having a first diameter; (ii) a suspension including: a liquid carrier; and a plurality of functionalized nanostructures dispersed throughout the liquid carrier, wherein each of the plurality of functionalized nanostructures has a second diameter that is equal to or less than the first diameter, and each of the plurality of functionalized nanostructures includes: a nanostructure core, and a plurality of primers attached to the nanostructure core; and (iii) a plurality of mechanical loading beads having a third diameter that is greater than the first diameter.

Each of the functionalized nanostructures includes a surface chemistry for seeding and clustering library templates.

### Functionalized Nanostructure

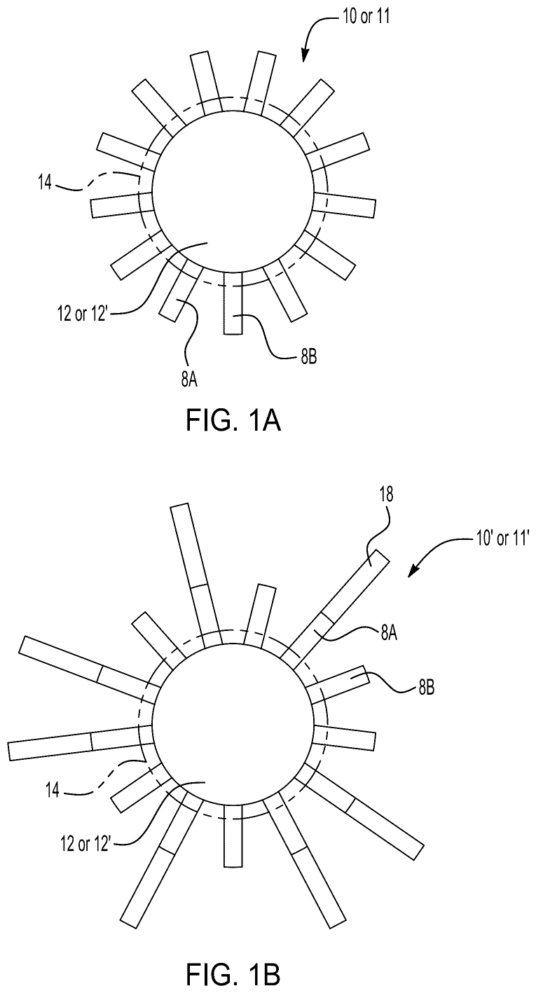

The functionalized nanostructures that are used in the kit may be pre-clustered, or non-pre-clustered. Examples of the non-pre-clustered functionalized nanostructures 10, 11 are shown in FIG. 1A, and examples of the pre-clustered functionalized nanostructures 10′, 11′ are shown in FIG. 1B.

Referring specifically to FIG. 1A, two examples of the non-pre-clustered functionalized nanostructure 10, 11 are depicted. In one example, the functionalized nanostructure 11 includes a hydrogel nanostructure core 12′ and a plurality of primers 8A, 8B attached to the hydrogel nanostructure core 12′. In another example, the functionalized nanostructure 10 includes a nanostructure core 12, a hydrogel coating 14 attached to the nanostructure core 12, and a plurality of primers 8A, 8B attached to side chains or arms of the hydrogel coating 14. The non-pre-clustered functionalized nanostructures 10, 11 may be introduced into the flow cell, and then on-board amplification may take place (as described in more detail herein).

Alternatively, the functionalized nanostructures 10, 11 may be used in off-board amplification techniques, in which amplicons (also referred to herein as template nucleic acid strands 18) become attached to the primers 8A, 8B before the functionalized nanostructure 10, 11 is introduced into the flow cell. Attachment of the template nucleic acid strands 18 to the primers 8A, 8B and subsequent amplification generates the pre-clustered functionalized nanostructure 10′, 11′ shown in FIG. 1B. The pre-clustered functionalized nanostructure 10′. 11′ may then be introduced into the flow cell for use in sequencing operations.

As mentioned, some examples of the functionalized nanostructure 10 include the nanostructure core 12, the hydrogel coating 14 attached to the nanostructure core 12, and the plurality of primers 8A, 8B attached to side chains or arms of the hydrogel coating 14.

In these examples, the material making up the nanostructure core 12 is generally rigid and is insoluble in an aqueous liquid. For example, the nanostructure core 12 can be inert to chemistry used to attach the primer(s) 8A, 8B, used in sequencing reactions, etc. Examples of suitable core 12 materials include magnetic materials (e.g., magnetic FeOₓ, silica coated FeOₓ), plastics (e.g., polytetrafluoroethylene (PTFE), some polyacrylics, polypropylene, polyethylene, polybutylene, polyurethanes, polystyrene and other styrene copolymers), nylon (i.e., polyamide), polycaprolactone (PCL), nitrocellulose, silica (SiO₂), silica-based materials (e.g., functionalized SiO₂), carbon, or metals.

As mentioned, in some examples, the nanostructure core 12 supports the hydrogel coating 14. In other examples, the hydrogel core 12′ is made up of the same type of hydrogel material used for the coating 14. In other words, the entire core 12′ is formed of the hydrogel material. In either example, the hydrogel material is a polymeric hydrogel. The polymeric hydrogel refers to a semi-rigid polymer that is permeable to liquids and gases. The polymeric hydrogel can swell when liquid (e.g., water) is taken up and that can contract when liquid is removed, e.g., by drying. A hydrogel material may absorb water while not being itself water-soluble.

Methods for forming the hydrogel core 12′ and for coating the hydrogel coating 14 on the nanostructure core 12 are described in more detail below.

In some examples, the polymeric hydrogel material is poly(N-(5-azidoacetamidylpentyl) acrylamide-co-acrylamide (PAZAM, as described below) or another of the acrylamide copolymers disclosed herein, poly(ethylene glycol) (PEG)-acrylate, PEG-diacrylate, PEG-amine, PEG-carboxylate, PEG-dithiol, PEG-epoxide, PEG-isocyanate, PEG-maleimide, crosslinked poly(methyl methacrylate) (PMMA), polyvinylpyrrolidone (PVPON), polyvinyl alcohol (PVA), polyethylene oxide-polypropylene oxide block copolymers (PEO-PPO), poly(hydroxyethyl methacrylate) (PHEMA), poly(N-isopropylacrylamide) (PNIPAAm), poly(lactic acid)-poly(ethylene glycol) block copolymers, poly(ethylene glycol)-poly(lactic-co-glycolic acid) block copolymers, poly(acrylic-co-vinylsulfonic acid), poly(acrylamide-co-vinylsulfonic acid), poly(L-aspartic acid), poly(aspartamide), adipic dihydrazide modified or aldehyde modified poly(L-glutamic acid), bisacrylamide, or hydrogels based on one or more of polylysine, starch, agar, agarose, heparin, alginate, alginate sulfate, dextran sulfate, hyaluronan, pectin, carrageenan, gelatin, chitosan, cellulose, and collagen, or combinations or mixtures thereof.

As described, poly(N-(5-azidoacetamidylpentyl)) acrylamide-co-acrylamide, referred to herein as “PAZAM,” is one example of the hydrogel coating 14 or hydrogel core 12′. PAZAM and some other forms of the acrylamide copolymer are represented by the following structure (I):

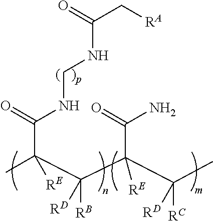

wherein:
R^{A} is selected from the group consisting of azido, optionally substituted amino, optionally substituted alkenyl, optionally substituted alkyne, halogen, optionally substituted hydrazone, optionally substituted hydrazine, carboxyl, hydroxy, optionally substituted tetrazole, optionally substituted tetrazine, nitrile oxide, nitrone, sulfate, and thiol;
R^{B} is H or optionally substituted alkyl;
R^{C}, R^{D}, and R^{E} are each independently selected from the group consisting of H and optionally substituted alkyl;
each of the —(CH₂)ₚ— can be optionally substituted;
p is an integer in the range of 1 to 50;
n is an integer in the range of 1 to 50,000; and
m is an integer in the range of 1 to 100,000.

One of ordinary skill in the art will recognize that the arrangement of the recurring “n” and “m” features in structure (I) are representative, and the monomeric subunits may be present in any order in the polymer structure (e.g., random, block, patterned, or a combination thereof).

The molecular weight of PAZAM and other forms of the acrylamide copolymer may range from about 5 kDa to about 1500 kDa or from about 10 kDa to about 1000 kDa, or may be, in a specific example, about 312 kDa.

In some examples, PAZAM and other forms of the acrylamide copolymer are linear polymers. In some other examples, PAZAM and other forms of the acrylamide copolymer are lightly cross-linked polymers.

In some examples, the gel material may be a variation of structure (I). In one example, the acrylamide unit may be replaced with N,N-dimethylacrylamide

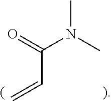

In another example, the acrylamide unit in structure (I) may be replaced with,

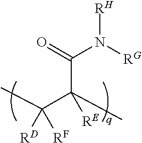

where R^{D}, R^{E}, and R^{F} are each H or a C1-C6 alkyl, and R^{G} and R^{H} are each a C1-C6 alkyl (instead of H as is the case with the acrylamide). In this example, q may be an integer in the range of 1 to 100,000. In another example, the N, N-dimethylacrylamide may be used in addition to the acrylamide unit. In this example, structure (I) may include

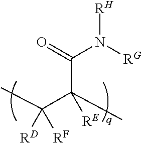

in addition to the recurring “n” and “m” features, where R^{D}, R^{E}, and R^{F} are each H or a C1-C6 alkyl, and R^{G} and R^{H} are each a C1-C6 alkyl. In this example, q may be an integer in the range of 1 to 100,000.

As another example, the recurring “n” feature in structure (I) may be replaced with a monomer including a heterocyclic azido group having structure (II):

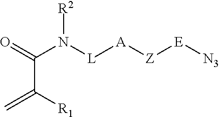

wherein R₁ is H or a C1-C6 alkyl; R² is H or a C1-C6 alkyl; L is a linker including a linear chain with 2 to 20 atoms selected from the group consisting of carbon, oxygen, and nitrogen and 10 optional substituents on the carbon and any nitrogen atoms in the chain; E is a linear chain including 1 to 4 atoms selected from the group consisting of carbon, oxygen and nitrogen, and optional substituents on the carbon and any nitrogen atoms in the chain; A is an N substituted amide with an H or a C1-C4 alkyl attached to the N; and Z is a nitrogen containing heterocycle. Examples of Z include 5 to 10 carbon-containing ring members present as a single cyclic structure or a fused structure. Some specific examples of Z include pyrrolidinyl, pyridinyl, or pyrimidinyl.

As still another example, the hydrogel coating 14 or hydrogel core 12′ may include a recurring unit of each of structure (III) and (IV):

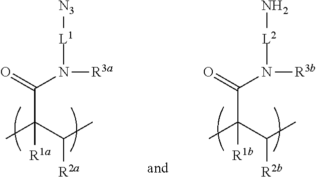

wherein each of R^{1a}, R^{2a}, R^{1b} and R^{2b} is independently selected from hydrogen, an optionally substituted alkyl or optionally substituted phenyl; each of R^{3a} and R^{3b} is independently selected from hydrogen, an optionally substituted alkyl, an optionally substituted phenyl, or an optionally substituted C7-C14 aralkyl; and each L¹ and L² is independently selected from an optionally substituted alkylene linker or an optionally substituted heteroalkylene linker.

In further examples, the polymeric hydrogel coating 14 or the hydrogel core 12′ is an alginate, acrylamide, or a PEG based material disclosed herein. In some examples, the polymeric hydrogel 14 or the hydrogel core 12′ is a PEG-based material with acrylate-dithiol, or epoxide-amine reaction chemistries. In some examples, the polymeric hydrogel coating 14 forms a polymer shell that includes PEG-maleimide/dithiol oil, PEG-epoxide/amine oil, PEG-epoxide/PEG-amine, or PEG-dithiol/PEG-acrylate.

Still further examples of suitable polymeric materials for the hydrogel coating 14 or hydrogel core 12′ include functionalized polysilanes, such as norbornene silane, azido silane, alkyne functionalized silane, amine functionalized silane, maleimide silane, or any other polysilane having functional groups that can attach the oligonucleotide primers 8A, 8B. Other examples of suitable hydrogel materials for the hydrogel coating 14 or the hydrogel core 12′ include those having a colloidal structure, such as agarose; or a polymer mesh structure, such as gelatin; or a cross-linked polymer structure, such as polyacrylamide polymers and copolymers, silane free acrylamide (SFA), or an azidolyzed version of SFA. Examples of suitable polyacrylamide polymers may be synthesized from acrylamide and an acrylic acid or an acrylic acid containing a vinyl group, or from monomers that form [2+2]photo-cycloaddition reactions. Still other examples of suitable polymeric hydrogel materials include mixed copolymers of acrylamides and acrylates. A variety of polymer architectures containing acrylic monomers (e.g., acrylamides, acrylates etc.) may be utilized in the examples disclosed herein, such as highly branched polymers, including dendrimers. For example, the monomers (e.g., acrylamide, etc.) may be incorporated, either randomly or in block, into the branches (arms) of a dendrimer.

An example of the dendrimeric polymeric hydrogel material includes a dendritic core with recurring units of formulas (II) and (III) in the arms extending from the dendritic core. The dendritic core may have anywhere from 3 arms to 30 arms.

The dendritic core may be any multi-functional component that enables a controlled polymerization mechanism, which leads to a defined arm length in the polymer structure and an at least substantially uniform arm length between polymer structures. In an example, the arms of the dendritic core are identical to each other.

The central molecule/compound of the dendritic core may be any multi-functional molecule, such as macrocycles (e.g., cyclodextrins, porphyrins, etc.), extended pi-systems (e.g., perylenes, fullerenes, etc.), metal-ligand complexes, polymeric cores, etc. Some specific examples of the central molecule/compound of the dendritic core include a phenyl group, benzoic acid, pentraerythritol, a phosphazene group, etc.

The dendritic core includes arms that extend from the central molecule/compound. Each arm may include a group that enables the monomers of formula (II) and (III) to be incorporated. In one example, a thiocarbonylthio group is included in each arm, and thus includes a reversible addition-fragmentation chain transfer agent (a RAFT agent). In another example, the dendritic core includes an atom transfer radical polymerization (ATRP) initiator in each arm. In still another example, the dendritic core includes a nitroxide (aminooxyl) mediated polymerization (NMP) initiator in each arm.

Functional groups in one or more of the recurring units of the hydrogel material of the hydrogel coating 14 or the hydrogel core 12′ are capable of attaching the primers 8A, 8B. These functional groups (e.g., R² in formula (I), NH₂, N₃, etc.) may be located in the side chains of the linear or branched polymeric hydrogel material. As noted, one example of the branched polymeric hydrogel material is a dendrimer, and in an example, the primer-grafting functional groups are located in each of the arms of the dendrimer. These functional groups may be introduced as part of the monomer(s) used in copolymerization. To control the number of primer 8A, 8B anchorage points, the monomer bearing the functional group may be increased or decreased. These functional groups may alternatively be introduced after copolymerization.

Other hydrogel materials may be used for the hydrogel coating 14, provided that these materials are functionalized to graft oligonucleotide primers 8A, 8B thereto and are capable of attaching to the nanostructure core 12. It is also to be understood that other hydrogel materials may be used for the hydrogel core 12′, as long as they are functionalized to graft oligonucleotide primers 8A, 8B thereto.

Polymeric hydrogel coatings 14 or the hydrogel core 12′ may be prepared by cross-linking hydrophilic biopolymers or synthetic polymers or polymerizing suitable monomers and then cross-linking the resulting polymer. Thus, in some examples, the hydrogel coating 14 or the hydrogel core 12′ may include a crosslinker. As used herein, the term “crosslinker” refers to a molecule that can form a three-dimensional network when reacted with the appropriate base monomers. Examples of the previously listed hydrogel polymers may include one or more crosslinkers, such as N,N′-bis(acryloyl) cystamine, diamines, dopamine, cysteamine, and aminosilanes. In some examples, a crosslinker forms a disulfide bond in the hydrogel polymer, thereby linking hydrogel polymers.

In some examples, each of the plurality of functionalized nanostructures 10, 10′ further includes silane at a surface of the nanostructure core 12; and the polymeric hydrogel coating 14 attached to the silane. Silanization of the nanostructure core 12 may be achieved by immersing the nanostructure core 12 in a silanizing solution including a silane (e.g., trimethoxysilane) and a suitable organic solvent.

In the functionalized nanostructure 10, the thickness of the hydrogel coating 14 on the nanostructure core 12 ranges from about 10 nm to about 200 nm. The hydrogel coating 14 can be in a dry state or can be in a swollen state, where it uptakes liquid. The 10 nm thickness represents the hydrogel coating 14 in the fully dry state, and the 200 nm thickness represents the hydrogel coating 14 in the fully swollen state.

The weight average molecular weight of the hydrogel material used for the hydrogel coating 14 or the hydrogel core 12′ (linear or branched) ranges from about 10 kDa to about 2,000 kDa. In other examples, the weight average molecular weight ranges from about 100 kDa to about 400 kDa. Increasing the molecular weight will increase the thickness of the hydrogel coating 14. For the dendrimer version of the hydrogel coating 14, the branching number may also be used to achieve the desired thickness. Increasing the branching number will also increase the thickness of the hydrogel coating 14. In an example, the branching number ranges from 3 to 30.

As is described in more detail in regard to FIG. 3B herein, the functionalized nanostructure 10, 10′, 11, 11′ has a diameter D₂ (referred to herein as a “second diameter”) that is smaller than a diameter of each of a plurality of depressions that is included in the flow cell. In examples, the second diameter D₂ (e.g., of the functionalized nanostructures 10, 10′, 11, or 11′) ranges from about 100 nm to about 1000 nm. In other examples, the second diameter D₂ ranges from about 225 nm to about 875 nm, or from about 250 nm to about 550 nm, or from about 275 nm to about 325 nm, or from about 290 nm to about 310 nm. These ranges may reflect the diameter of the functionalized nanostructure 10, 10′, 11, 11′ when the hydrogel coating 14 or hydrogel core 12′ is in a swollen state.

The functionalized nanostructures 10, 10′, 11, 11′ also include the primers 8A, 8B. The polymeric hydrogel coating 14 over the core 12 or the hydrogel core 12′ provides a surface for attachment of the primers 8A, 8B.

The primer set attached to the polymeric hydrogel coating 14 or the hydrogel core 12′ includes two different primers 8A, 8B that are used in sequential paired end sequencing. As examples, the primer set may include P5 and P7 primers, P15 and P7 primers, or any combination of the PA primers, the PB primers, the PC primers, and the PD primers set forth herein. As examples, the primer set may include any two PA, PB, PC, and PD primers, or any combination of one PA primer and one PB, PC, or PD primer, or any combination of one PB primer and one PC or PD primer, or any combination of one PC primer and one PD primer. Examples of P5 and P7 primers are used on the surface of commercial flow cells sold by Illumina Inc. for sequencing, for example, on HISEQ™, HISEQX™, MISEQ™, MISEQDX™, MINISEQ™, NEXTSEQ™, NEXTSEQDX™, NOVASEQ™, NOVASEQX™, GENOME ANALYZER™, ISEQ™, and other instrument platforms. The P5 and P7 primers have a universal sequence for seeding and/or amplification purposes.

The P5 primer (which may be a cleavable primer due to the cleavable nucleobase uracil or “n”) is:

P5 #1: 5′ → 3′
(SEQ. ID. NO. 1)
AATGATACGGCGACCACCGAGAUCTACAC;
P5 #2: 5′ → 3′
(SEQ. ID. NO. 2)
AATGATACGGCGACCACCGAGAnCTACAC

where “n” is inosine in SEQ. ID. NO. 2; or

P5 #3: 5′ → 3′
(SEQ. ID. NO. 3)
AATGATACGGCGACCACCGAGAnCTACAC

where “n” is alkene-thymidine (i.e., alkene-dT) in SEQ. ID. NO. 3.
The P7 primer (which may be a cleavable primer) may be any of the following:

P7 #1: 5′ → 3′
(SEQ. ID. NO. 4)
CAAGCAGAAGACGGCATACGAnAT

where “n” is 8-oxoguanine in SEQ. ID. NO. 4;

P7 #2: 5′ → 3′
(SEQ. ID. NO. 5)
CAAGCAGAAGACGGCATACnAGAT

where “n” is 8-oxoguanine in SEQ. ID. NO. 5;

P7 #3: 5′ → 3′
(SEQ. ID. NO. 6)
CAAGCAGAAGACGGCATACnAnAT

where both instances of “n” are 8-oxoguanine in SEQ. ID. NO. 6;

P7 #4: 5′ → 3′
(SEQ. ID. NO. 7)
CAAGCAGAAGACGGCATACGAUAT;
or
P7 #5: 5′ → 3′
(SEQ. ID. NO. 8)
CAAGCAGAAGACGGCATACUAGAT.

The P15 primer (shown as a cleavable primer) is:

P15: 5′ → 3′
(SEQ. ID. NO. 9)
AATGATACGGCGACCACCGAGAnCTACAC

where “n” is allyl-T (i.e., a thymine nucleotide analog having an allyl functionality).
The other primers (PA-PD, shown as non-cleavable primers) mentioned above include:

PA 5′ → 3′
(SEQ. ID. NO. 10)
GCTGGCACGTCCGAACGCTTCGTTAATCCGTTGAG;
PB 5′ → 3′
(SEQ. ID. NO. 11)
CGTCGTCTGCCATGGCGCTTCGGTGGATATGAACT;
PC 5′ → 3′
(SEQ. ID. NO. 12)
ACGGCCGCTAATATCAACGCGTCGAATCCGCAACT;
and
PD 5′ → 3′
(SEQ. ID. NO. 13)
GCCGCGTTACGTTAGCCGGACTATTCGATGCAGC.

While not shown in the example sequences for PA-PD, it is to be understood that any of these primers may include a cleavage site, such as uracil, 8-oxoguanine, allyl-T, etc. at any point in the strand.

It is to be understood that the cleavage sites of the primers 8A, 8B in the primer set are orthogonal to each other (i.e., one cleavage site is not susceptible to the cleaving agent used for the other cleavage site), so that after amplification, forward or reverse strands can be cleaved, leaving the other of the reverse or forward strands for sequencing.

Each of the primers 8A, 8B disclosed herein may also include a polyT sequence at the 5′ end of the primer sequence. In some examples, the polyT region includes from 2 T bases to 20 T bases. As specific examples, the polyT region may include 3, 4, 5, 6, 7, or 10 T bases.

The 5′ end of each primer 8A, 8B may also include a linker. Any linker that includes a terminal alkyne group or another suitable terminal functional group that can attach to the surface functional groups of the hydrogel 14 or the hydrogel core 12′ may be used. In one example, 5′ end of the primers 8A, 8B are terminated with a hexynyl functionality.

The immobilization of the primers 8A, 8B may be by single point covalent attachment at the 5′ end of the primers 8A, 8B. The attachment will depend, in part, on the functional groups of the hydrogel coating 14 or the hydrogel core 12′. Examples of terminated primers that may be used include an alkyne terminated primer, a tetrazine terminated primer, an azido terminated primer, an amino terminated primer, an epoxy or glycidyl terminated primer, a thiophosphate terminated primer, a thiol terminated primer, an aldehyde terminated primer, a hydrazine terminated primer, a phosphoramidite terminated primer, and a triazolinedione terminated primer. As specific examples, a succinimidyl (NHS) ester terminated primer may be reacted with an amine of the hydrogel coating 14 or the hydrogel core 12′, an aldehyde terminated primer may be reacted with a hydrazine of the hydrogel coating 14 or the hydrogel core 12′, an alkyne terminated primer may be reacted with an azide of the hydrogel coating 14 or the hydrogel core 12′, an azide terminated primer may be reacted with an alkyne or DBCO (dibenzocyclooctyne) of the hydrogel coating 14 or the hydrogel core 12′, an amino terminated primer may be reacted with an activated carboxylate group or NHS ester of the hydrogel coating 14 or the hydrogel core 12′, a thiol terminated primer may be reacted with an alkylating reactant (e.g., iodoacetamine or maleimide) of the hydrogel coating 14 or the hydrogel core 12′, or a phosphoramidite terminated primer may be reacted with a thioether of the hydrogel coating 14 or the hydrogel core 12′. While several examples have been provided, it is to be understood that a functional group that can be attached to the primer 8A, 8B and that can attach to a functional group of the hydrogel coating 14 or the hydrogel core 12′ may be used.

In a specific example, each of the plurality of functionalized nanostructures 10, 10′ further includes a polymeric hydrogel coating 14 attached to the nanostructure core 12; each of the plurality of primers 8A, 8B is attached to a side chain or arm of the polymeric hydrogel coating 14; and each of the plurality of primers 8A, 8B is functionalized with an azide group or an alkyne group.

The functionalized nanostructure 10, 10′, 11, or 11′ may be loaded into depressions of a flow cell. The structure of the flow cell will now be described.

### Flow Cell

FIG. 2A depicts an example of the flow cell 20 disclosed herein from a top view, and an example of an architecture within a flow channel 21 of the flow cell 20 is shown in FIG. 2B. Examples of enclosed flow cells 20 may include one patterned structure 24A or 24B bonded to a lid (lid not shown) or two patterned structures 24A and 24B (see FIG. 3A through FIG. 3C) bonded together. Another example of the flow cell 20 is an open-wafer flow cell that includes a single patterned structure 24A that is open to the surrounding environment.

The flow channel 21 in the enclosed form of the flow cells 20 is defined between the one patterned structure 24A and the lid or the second patterned structure 24B, which are bonded together via a spacer layer 34 (see FIG. 3A). Thus, each flow channel 21 in the enclosed form of the flow cells 20 is defined by the patterned structure 24A, the spacer layer 34, and either the lid or the second patterned structure 24B. Alternatively, when a single patterned structure 24A or 24B is used (e.g., as an open-wafer substrate), the flow channel 21 may be defined by a lane that has been patterned into the substrate 24A (e.g., via nanolithography) and in which the depressions 22 are formed. In this example, the depth of the lane is greater than the depth of the depressions 22 defined within the lane.

The example flow cell 20 shown in FIG. 2A includes eight flow channels 21. While eight flow channels 21 are shown, it is to be understood that any number of flow channels 21 may be included in the flow cell 20 (e.g., a single flow channel 21, four flow channels 21, etc.). When multiple flow channels 21 are included, each flow channel 21 may be isolated from another flow channel 21 so that fluid introduced into a flow channel 21 does not flow into (an) adjacent flow channel(s) 21.

Each flow channel 21 is in fluid communication with an inlet and an outlet (not shown). The inlet and outlet of each flow channel 21 may be positioned at opposed ends of the flow cell 20. The inlets and outlets of the respective flow channels 21 may alternatively be positioned anywhere along the length and width of the flow channel 21 that enables desirable fluid flow.

The inlet allows fluids to be introduced into the flow channel 21, and the outlet allows fluid to be extracted from the flow channel 21. Each of the inlets and outlets is fluidly connected to a fluidic control system (including, e.g., reservoirs, pumps, valves, waste containers, and the like) which controls fluid introduction and expulsion. Some examples of the fluids introduced into the flow channel 21 may introduce the suspension disclosed herein (e.g., the suspension including the liquid carrier and the plurality of functionalized nanostructures 10, 10′, 11, 11′ disclosed herein), reaction components (e.g., DNA sample, polymerases, sequencing primers, nucleotides, etc.), washing solutions, deblocking agents, etc.

The flow channel 21 may have any desirable shape. In an example, the flow channel 21 has a substantially rectangular configuration with curved ends (as shown in FIG. 2A). The length of the flow channel 21 depends, in part, upon the size of the substrate (e.g., 16 or 26, see FIG. 2B) used to form the patterned structure 24A or 24B. The width of the flow channel 21 depends, in part, upon the size of the substrate 16 or 26 used to form the patterned structure 24A or 24B, the desired number of flow channels 21, the desired space between adjacent channels 21, and the desired space at a perimeter of the patterned structure 24A or 24B. The spaces between channels 21 and at the perimeter of the patterned structure 24A may be sufficient for attachment to a lid (not shown) or another patterned structure 24B.

The depth of the flow channel 21 can be as small as a monolayer thick when microcontact, aerosol, or inkjet printing is used to deposit a separate material (e.g., the spacer layer) that defines at least a portion of the sidewalls of the flow channel 21. For other examples, the depth of the flow channel 21 can be about 1 μm, about 10 μm, about 50 μm, about 100 μm, or more. In an example, the depth may range from about 10 μm to about 100 μm. In another example, the depth may range from about 10 μm to about 30 μm. In still another example, the depth is about 5 μm or less. It is to be understood that the depth of the flow channel 21 may be greater than, less than or between the values specified above.

The spacer layer 34 used to attach the patterned structure 24A and the lid or the second patterned structure 24B may be any material that will seal portions of the patterned structure 24A and the lid or the second patterned structure 24B. As examples, the spacer layer 34 may be an adhesive, a radiation-absorbing material that aids in bonding, or the like. In some examples, the spacer layer 34 is the radiation-absorbing material, e.g., KAPTON® black.

The patterned structure 24A and the lid or the second patterned structure 24B may be bonded using any suitable technique, such as laser bonding, diffusion bonding, anodic bonding, eutectic bonding, plasma activation bonding, glass frit bonding, or others methods known in the art.

When used, the lid may be any material that is transparent to the excitation light that is directed toward the flow cell 20 (e.g., during a sequencing operation, a polymerization step, etc.). In optical detection systems, the lid may also be transparent to the emissions generated from reaction(s) taking place in the flow cell 20. As examples, the lid may include glass (e.g., borosilicate, fused silica, etc.) or a transparent polymer. A commercially available example of a suitable borosilicate glass is D 263®, available from Schott North America, Inc. Commercially available examples of suitable polymer materials, namely cyclo olefin polymers, are the ZEONOR® products available from Zeon Chemicals L.P. In some instances, the lid is shaped to form the top of the flow cell 20, and in other instances, the lid is shaped to form both the top of the flow cell 20 as well as sidewalls the flow channel 21.

The patterned structure 24A includes a bonding region 23 where it can be sealed to the lid or to the second patterned structure 24B. The bonding region 23 may be located at the perimeter of each flow channel 21 (as shown in FIG. 2B) and at the perimeter of the flow cell 20.

The patterned structure 24A, 24B includes a substrate 16 or 26, as shown in FIG. 2B. The substrate 16 is a single layer substrate, and the substrate 26 has an additional layer 28 thereon (and thus the substrate 26 is part of a multi-layered structure). The substrate 16 is a single material that has depressions 22 defined therein. The substrate 26 includes a single material and further includes the layer 28 positioned thereon. The layer 28 includes a separate single material which may be different than the material of the substrate 26, and the layer 28 has the depressions 22 defined therein.

Examples of suitable materials for the substrate 16 include siloxanes (e.g., epoxy siloxane), glass, modified or functionalized glass, polystyrene and copolymers of styrene and other materials, polypropylene, polyethylene, polybutylene, polyurethanes, polytetrafluoroethylene (such as TEFLON® from Chemours), polyethylene terephthalate (PET), polycarbonate, cyclic olefins/cyclo-olefin polymers (COP) (such as ZEONOR® from Zeon), polyimides, nylon (polyamides), ceramics/ceramic oxides, silica (i.e., silicon dioxide (SiO₂)), fused silica, or silica-based materials, aluminum silicate, silicon and modified silicon (e.g., boron doped p+ silicon), silicon nitride (Si₃N₄), tantalum pentoxide (Ta₂O₅) or other tantalum oxide(s) (TaOₓ), hafnium oxide (HfO₂), carbon, metals, resins, or the like. Examples of suitable resins include inorganic oxides, such as tantalum pentoxide (e.g., Ta₂O₅) or other tantalum oxide(s) (TaOₓ), aluminum oxide (e.g., Al₂O₃), silicon oxide (e.g., SiO₂), hafnium oxide (e.g., HfO₂), indium tin oxide, titanium dioxide, etc., or polymeric resins, such as a polyhedral oligomeric silsesquioxane based resin (e.g., POSS® from Hybrid Plastics), a non-polyhedral oligomeric silsesquioxane epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof.

As mentioned, examples of the multi-layered structure include the substrate 26 and at least one other layer 28 thereon. Any example of the single layer substrate 16 may be used as the substrate 26 of the multi-layered structure. In these examples, the other layer 28 (on the substrate 26) may be any material that can be etched or imprinted to form the depressions 22. Examples of the layer 28 include inorganic oxides, such as tantalum oxide (e.g., Ta₂O₅), aluminum oxide (e.g., Al₂O₃), silicon oxide (e.g., SiO₂), or hafnium oxide (e.g., HfO₂), or polymeric resins, such as a polyhedral oligomeric silsesquioxane based resin (e.g., POSS® from Hybrid Plastics), a non-polyhedral oligomeric silsesquioxane epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof.

Suitable deposition techniques for the layer 28 or for the substrate 16 include dip coating, dunk coating, spin coating, spray coating, puddle dispensing, ultrasonic spray coating, doctor blade coating, aerosol printing, screen printing, microcontact printing, etc. In some instances in which the layer 28 or the substrate 16 includes a resin material, following deposition and patterning, the resin of the layer 28 or the substrate 16 may be cured, e.g., via exposure to actinic radiation or heat.

In any of the examples set forth herein, the substrate 16 or 26 may be a circular sheet, a panel, a wafer, a die, etc. having a diameter ranging from about 2 mm to about 300 mm, e.g., from about 200 mm to about 300 mm, or may be a rectangular sheet, panel, wafer, die etc. having its largest dimension up to about 10 feet (˜3 meters). As one example, a die may have a width ranging from about 0.1 mm to about 10 mm. While example dimensions have been provided, it is to be understood that the substrate 16 or 26 may have any suitable dimensions.

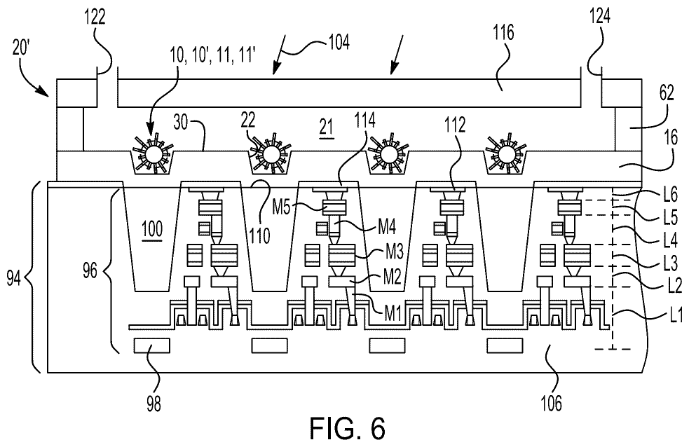

As shown in FIG. 6, one example of the flow cell 20′ is attached to a complementary metal oxide semiconductor (CMOS) chip 94. While the flow cell 20′ is shown as the enclosed version with a lid 116, it is to be understood that an open-wafer form of the flow cell 20′ may be attached to the CMOS chip 94.

In addition to the complementary metal oxide semiconductor chip 94, this example flow cell 20′ includes the substrate 16 over the complementary metal oxide semiconductor chip 94, the substrate 16 including a plurality of depressions 22 separated by interstitial regions 30; and a functionalized nanostructure 10, 10′, 11, or 11′ within the depressions 22. FIG. 6 depicts the substrate 16 coupled to the CMOS chip 94 for ease of illustration, but it is to be understood that the flow cell 20′ may alternatively include a substrate 26 having a layer 28 thereon, where the depressions 22 are defined in the layer 28 and the substrate 26 is coupled to the CMOS chip 94.

In the illustrated example, the substrate 16 of the flow cell 20′ may be affixed directly to, and thus be in physical contact with, the complementary metal oxide semiconductor chip 94 through one or more securing mechanisms (e.g., adhesive, bond, fasteners, and the like). It is to be understood that the substrate 16 may be removably coupled to the complementary metal oxide semiconductor (CMOS) chip 94.

The CMOS chip 94 includes a plurality of stacked layers 96 including, for example, silicon layer(s), dielectric layer(s), metal-dielectric layer(s), metal layer(s), etc.). The stacked layers 96 make up the device circuitry, which includes detection circuitry.

The CMOS chip 94 includes optical components, such as optical sensor(s) 98 and optical waveguide(s) 100. The optical components are arranged such that each optical sensor 98 at least substantially aligns with, and thus is operatively associated with, a single optical waveguide 100 and a single depression 22 of the flow cell 20′. However, in other examples, a single optical sensor 98 may receive photons through more than one optical waveguide 100 and/or from more than one depression 22. In these other examples, the single optical sensor 98 is operatively associated with more than one optical waveguide 100 and/or more than one depression 22.

As used herein, a single optical sensor 98 may be a light sensor that includes one pixel or more than one pixel. As an example, each optical sensor 98 may have a detection area that is less than about 50 μm². As another example, the detection area may be less than about 10 μm². As still another example, the detection area may be less than about 2 μm². In the latter example, the optical sensor 98 may constitute a single pixel. An average read noise of each pixel of the optical sensor 98 may be, for example, less than about 150 electrons. In other examples, the read noise may be less than about 5 electrons. The resolution of the optical sensor(s) 98 may be greater than about 0.5 megapixels (Mpixels). In other examples, the resolution may be greater than about 5 Mpixels, or greater than about 10 Mpixels.

Also as used herein, a single optical waveguide 100 may be a light guide including a cured filter material that i) filters the excitation light 104 (propagating from an exterior of the flow cell 20′ into the flow channel 21), and ii) permits the light emissions (not shown, resulting from reactions at the depressions 22) to propagate therethrough toward corresponding optical sensor(s) 98. In an example, the optical waveguide 100 may be, for example, an organic absorption filter. As a specific example, the organic absorption filter may filter excitation light 104 of about 532 nm wavelength and permit light emissions of about 570 nm or more wavelengths. The optical waveguide 100 may be formed by first forming a guide cavity in a dielectric layer 106, and then filling the guide cavity with a suitable filter material.

The optical waveguide 100 may be configured relative to the dielectric material 106 in order to form a light-guiding structure. For example, the optical waveguide 100 may have a refractive index of about 2.0 so that the light emissions are substantially reflected at an interface between the optical waveguide 100 and the surrounding dielectric material 106. In certain examples, the optical waveguide 100 is selected such that the optical density (OD) or absorbance of the excitation light 104 is at least about 4 OD. More specifically, the filter material may be selected and the optical waveguide 100 may be dimensioned to achieve at least 4 OD. In other examples, the optical waveguide 100 may be configured to achieve at least about 5 OD or at least about 6 OD.

The flow cell 20′ includes the substrate 16, which is positioned over and attached to the complementary metal oxide semiconductor chip 94. The substrate 16 may be any example disclosed herein, and includes a plurality of depressions 22 separated by interstitial regions 30; and a functionalized nanostructure 10, 10′, 11, 11′ within the depressions 22.

In this example, the substrate 16 functions as a passivation layer. At least a portion of the passivating substrate 16 is in contact with a first embedded metal layer 112 of the CMOS chip 94 and also with an input region 110 of the optical waveguide 100. The contact between the passivating substrate 16 and the first embedded metal layer 112 may be direct contact or may be indirect contact through a shield layer 114.

The substrate 16 (passivation layer) may provide one level of corrosion protection for the embedded metal layer 112 of the CMOS chip 94 that is closest in proximity to the substrate 16. In this example, the substrate 16 may include a passivation material that is transparent to the light emissions resulting from reactions within the depressions 22 (e.g., visible light), and that is at least initially resistant to the fluidic environment and moisture that may be introduced into or present in the flow channel 21. An at least initially resistant material acts as an etch barrier to high pH reagents (e.g., pH ranging from 8 to 14) and as a moisture barrier. Examples of suitable materials for the substrate 16 of the flow cell 20′ include silicon nitride (Si₃N₄), silicon oxide (SiO₂), tantalum pentoxide (TaO₅), hafnium oxide (HfO₂), boron doped p+ silicon, or the like. The thickness of the substrate 16 may vary depending, in part, upon the sensor dimensions. In an example, the thickness of the substrate 16 ranges from about 100 nm to about 500 nm.

In the open-wafer form, the substrate 16 includes a lane with sidewalls that create the flow channel 21. The depressions 22 are formed in the substrate 16 within the lane.

In the example shown in FIG. 6, the flow cell 20′ also includes a lid 116 that is operatively connected to the substrate 16 to partially define the flow channel 21 between the substrate 16 (and the depressions 22 therein) and the lid 116. The lid 116 may be any material that is transparent to the excitation light 104 that is directed toward the depressions 22. As examples, the lid 116 may include glass (e.g., borosilicate, fused silica, etc.), plastic, etc. A commercially available example of a suitable borosilicate glass is D 263®, available from Schott North America Inc. Commercially available examples of suitable plastic materials, namely cyclo olefin polymers, are the ZEONOR® products available from Zeon Chemicals L.P.

The lid 116 may be physically connected to the substrate 16 through material 62, which is similar to the spacer layer 34. The material 62 is/are coupled to a portion the surface of the substrate 16 and extends between that surface and an interior surface of the lid 116. In some examples, the material 62 and the lid 116 may be integrally formed, such that they 62, 116 are a continuous piece of material (e.g., glass or plastic). In other examples, the material 62 and the lid 116 may be separate components that are coupled to each other. In these other examples, the material 62 may be the same material as, or a different material than the lid 116. In some of these other examples, at least one of the materials 62 includes an electrode material. In still other examples, the material 62 includes a curable adhesive layer that bonds the lid 116 to the substrate 16 (at a portion of its surface).

In an example, the lid 116 may be a substantially rectangular block having an at least substantially planar exterior surface and an at least substantially planar interior surface that defines a portion of the flow channel 21. The block may be mounted onto the material 62. Alternatively, the block may be etched to define the lid 116 and the material 62 (which functions as sidewall(s)). For example, a recess may be etched into the transparent block. When the etched block is mounted to the substrate 16, the recess may become the flow channel 21.

The lid 116 may include inlet and outlet ports 122, 124 that are configured to fluidically engage other ports (not shown) for directing fluid(s) into the flow channel 21 (e.g., from a reagent cartridge or other fluid storage system component) and out of the flow channel 21 (e.g., to a waste removal system).

The flow channel 21 may be sized and shaped to direct a fluid along the depressions 22. The height of the flow channel 21 and other dimensions of the flow channel 21 may be configured to maintain a substantially even flow of the fluid over the depressions 22. The dimensions of the flow channel 21 may also be configured to control bubble formation. In an example, the height of the flow channel 21 may range from about 50 μm to about 400 μm. In another example, the height of the flow channel 21 may range from about 80 μm to about 200 μm. It is to be understood that the height of the flow channel 21 may vary.

Each depression 22 is a localized region in the substrate 16 where a designated reaction may occur. In the flow cell 20′, each depression 22 is shown as having a functionalized nanostructure 10, 10′, 11, or 11′ therein. The functionalized nanostructure 10, 10′, 11, or 11′ may be introduced into the depressions 22 using a plurality of mechanical loading beads (as described herein in regard to FIG. 3A through FIG. 3C).

In an example, each depression 22 is at least substantially aligned with the input region 110 of a single optical waveguide 100. As such, light emissions at the depressions 22 may be directed into the input region 110, through the waveguide 100, and to an associated optical sensor 98. In other examples, one depression 22 may be aligned with several input regions 110 of several optical waveguides 100. In still other examples, several depressions 22 may be aligned with one input region 110 of one optical waveguide 100.

The embedded metal layer 112 may be any suitable CMOS metal, such as aluminum (Al), aluminum chloride (AlCl), tungsten (W), nickel (Ni), or copper (Cu). The embedded metal layer 112 is a functioning part of the CMOS AVdd line, and through the stacked layers 96, is also electrically connected to the optical sensor 98. Thus, the embedded metal layer 112 participates in the detection/sensing operation.

It is to be understood that the other optical sensors 98 and associated components may be configured in an identical or similar manner. It is also to be understood, however, that the CMOS chip 94 may not be manufactured identically or uniformly throughout. Instead, one or more optical sensor 98 and/or associated components may be manufactured differently or have different relationships with respect to one another

The stacked layer 96 may include interconnected conductive elements (e.g., conductors, traces, vias, interconnects, etc.) that can conduct electrical current. The circuitry may be configured for selectively transmitting data signals that are based on detected photons. The circuitry may also be configured for signal amplification, digitization, storage, and/or processing. The circuitry may collect and analyze the detected light emissions and generate data signals for communicating detection data to a bioassay system. The circuitry may also perform additional analog and/or digital signal processing in the CMOS chip 94.

The CMOS chip 94 may be manufactured using integrated circuit manufacturing processes. The CMOS chip 94 may include multiple layers, such as a sensor base/layer (e.g., a silicon layer or wafer). The sensor base may include the optical sensor 98. When the CMOS chip 94 is fully formed, the optical sensor 98 may be electrically coupled to the rest of the circuitry in the stack layer 96 through gate(s), transistor(s), etc.

As used in reference to FIG. 6, the term “layer” is not limited to a single continuous body of material unless otherwise noted. For example, the sensor base/layer may include multiple sub-layers that are different materials and/or may include coatings, adhesives, and the like. Furthermore, one or more of the layers (or sub-layers) may be modified (e.g., etched, deposited with material, etc.) to provide the features described herein.

The stacked layer 96 also includes a plurality of metal-dielectric layers. Each of these layers includes metallic elements (e.g., M1-M5, which may be, for example, W (tungsten), Cu (copper), Al (aluminum), or any other suitable CMOS conductive material) and dielectric material 106 (e.g., SiO₂). Various metallic elements M1-M5 and dielectric materials 106 may be used, such as those suitable for integrated circuit manufacturing.

In the example shown in FIG. 6, each of the plurality of metal-dielectric layers L1-L6 includes both metallic elements M1, M2, M3, M4, M5 and dielectric material 106. In each of the layers L1-L6, the metallic elements M1, M2, M3, M4, M5 are interconnected and are embedded within dielectric material 106. In some of the metal-dielectric layers L1-L6, additional metallic elements may also be included. Some of these additional metallic elements may be used to address individual pixels through a row and column selector. The voltages at these elements may vary and switch between about-1.4 V and about 4.4 V depending upon which pixel the device is reading out.

The configuration of the metallic elements M1, M2, M3, M4, M5 and dielectric layer 106 in FIG. 6 is illustrative of the circuitry, and it is to be understood that other examples may include fewer or additional layers and/or may have different configurations of the metallic elements M1-M5.

In the example shown in FIG. 6, the shield layer 114 is in contact with at least a portion of the substrate 16. The shield layer 114 has an aperture at least partially adjacent to the input region 110 of the optical waveguide 100. This aperture enables the depressions 22 (and at least some of the light emissions therefrom) to be optically connected to the waveguide 100. It is to be understood that the shield layer 114 may have an aperture at least partially adjacent to the input region 110 of each optical waveguide 100. The shield layer 114 may extend continuously between adjacent apertures.

The shield layer 114 may include any material that can block, reflect, and/or significantly attenuate the light signals that are propagating through the flow channel 21. The light signals may be the excitation light 104 and/or the light emissions from the depressions 22. As an example, the shield layer 114 may be tungsten (W).

It is to be understood that the flow cell 20′ may also be used for optical detection.

Returning now to FIG. 2B, the substrate 16 or the layer 28 includes the plurality of depressions 22 defined therein. In some examples (and as shown in FIG. 2B), each of the plurality of depressions 22 is separated from each other of the plurality of depressions 22 by interstitial regions 30.

The depressions 22 may be formed in the layer 28 or in the substrate 16 using any suitable technique, such as nanoimprint lithography or photolithography. For example, a working stamp including a negative replica of the depressions 22 may be pressed into the layer 28 or the substrate 16 (e.g., when the layer 28 or the substrate 16 includes a resin) while the layer 28 or substrate 16 is soft. Curing of the resin may then be performed, e.g., via actinic radiation or heat, with the working stamp in place. Release of the working stamp forms the depressions 22 in the substrate 16 or the layer 28.

Many different layouts of the depressions 22 may be envisaged, including regular, repeating, and non-regular patterns. In an example, the depressions 22 are disposed in a hexagonal grid for close packing and improved density. Other layouts may include, for example, rectangular layouts, triangular layouts, and so forth. In some examples, the layout or pattern can be an x-y format in rows and columns. In some other examples, the layout or pattern can be a repeating arrangement of the depressions 22 and interstitial regions 30. In still other examples, the layout or pattern can be a random arrangement of the depressions 22 and the interstitial regions 30.

The layout or pattern may be characterized with respect to the density (number) of the depressions 22 in a defined area. For example, the depressions 22 may be present at a density of approximately 2 million per mm². The density may be tuned to different densities including, for example, a density of about 100 per mm², about 1,000 per mm², about 0.1 million per mm², about 1 million per mm², about 2 million per mm², about 5 million per mm², about 10 million per mm², about 50 million per mm², or more, or less. It is to be further understood that the density can be between one of the lower values and one of the upper values selected from the ranges above, or that other densities (outside of the given ranges) may be used. As examples, a high density array may be characterized as having the depressions 22 separated by less than about 100 nm, a medium density array may be characterized as having the depressions 22 separated by about 400 nm to about 1 μm, and a low density array may be characterized as having the depressions 22 separated by greater than about 1 μm.

The layout or pattern of the depressions 22 may also or alternatively be characterized in terms of the average pitch, or the spacing from the center of one depression 22 to the center of an adjacent depression 22 (center-to-center spacing) or from the right edge of one depression 22 to the left edge of an adjacent depression 22. The pattern can be regular, such that the coefficient of variation around the average pitch is small, or the pattern can be non-regular in which case the coefficient of variation can be relatively large. In either case, the average pitch can be, for example, about 50 nm, about 0.15 μm, about 0.5 μm, about 1 μm, about 5 μm, about 10 μm, about 100 μm, or more or less. The average pitch for a particular pattern of depressions 22 can be between one of the lower values and one of the upper values selected from the ranges herein. In an example, the depressions 22 have a pitch (center-to-center spacing) of about 1.5 μm. While example average pitch values have been provided, it is to be understood that other average pitch values may be used.

The size of each depression 22 may be characterized by its volume, opening area, depth, and/or diameter or length and width. For example, the volume can range from about 1×10⁻³ μm³ to about 100 μm³, e.g., about 1×10⁻² μm³, about 0.1 μm³, about 1 μm³, about 10 μm³, or more, or less. For another example, the opening area can range from about 1×10−3 μm² to about 100 μm², e.g., about 1× 10-2 μm², about 0.1 μm², about 1 μm², at least about 10 μm², or more, or less. For still another example, the depth can range from about 0.1 μm to about 100 μm, e.g., about 0.5 μm, about 1 μm, about 10 μm, or more, or less. For another example, the depth can range from about 0.1 μm to about 100 μm, e.g., about 0.5 μm, about 1 μm, about 10 μm, or more, or less. For yet another example, the diameter or each of the length and width can range from about 0.1 μm to about 100 μm, e.g., about 0.5 μm, about 1 μm, about 10 μm, or more, or less.

Further, each of the plurality of depressions 22 has a diameter D₁, as shown in FIG. 3B (referred to herein as a “first” diameter D₁). It is to be understood that the second diameter D₂ (e.g., of each of the plurality of functionalized nanostructures 10, 10′, 11, 11′ described herein) is less than or equal to the first diameter D₁. As such, the depressions 22 can spatially accommodate the functionalized nanostructures 10, 10′, 11, 11′.

In an example, the first diameter D₁ ranges from about 250 nm to about 1000 nm. In further examples, the first diameter D₁ or from about 325 nm to about 725 nm, or from about 350 nm to about 400 nm, or from about 300 nm to about 600 nm. In a specific example, the first diameter D₁ is about 360 nm.

As shown in FIG. 2B, each of the plurality of depressions 22 includes at least one functionalized nanostructure 10, 10′, 11, 11′ therein. When the functionalized nanostructures 10, 10′, 11, 11′ are loaded into the flow cell 20 using the method disclosed herein, it is believed that greater than 90% loading occupancy of the nanostructures 10, 10′, 11, 11′ can be achieved. Depending on the first diameter D₁ of each of the plurality of depressions 22 and on the second diameter D₂ of each of the plurality functionalized nanostructures 10, 10′, 11, 11′, in some examples, more than one functionalized nanostructure 10, 10′, 11, 11′ may be included in each depression 22 (as shown in the center depression 22 of FIG. 2B).

The plurality of functionalized nanostructures 10, 10′, 11, 11′ may be introduced into the depressions 22 of the flow cell 20 by (i) including the nanostructures 10, 10′, 11, 11′ in the suspension including the liquid carrier, where the suspension is to be introduced into the flow cell 20 via an inlet, and (ii) using a plurality of mechanical loading beads (not shown in FIG. 2B). The liquid carrier and the loading beads will now be described.

### Liquid Carrier

The liquid carrier may be any suitable liquid carrier that can be introduced into a flow cell 20, 20′ and that can be used to suspend the functionalized nanostructure(s) 10, 10′, 11, or 11′. In an example, the liquid carrier is an aqueous solution including a buffer and/or a polar aprotic solvent. Suitable buffers include phosphate buffers and suitable polar aprotic solvents include acetone, chloroform, and dichloromethane. In another example, the liquid carrier also includes formamide.

The amount, or concentration, of functionalized nanostructures 10, 10′, 11, 11′ suspended in the liquid carrier may depend, in part, upon the size (e.g., D₂) of each of the functionalized nanostructures 10, 10′, 11, 11′ and the density of the depressions 22. As an example, when the second diameter D₂ of each of the functionalized nanostructures 10, 10′, 11, 11′ is greater than about 200 nm, a loading of 0.1 mg (of nanostructures 10, 10′, 11, 11′) per mL of liquid carrier may be used. In this example, the concentration of functionalized nanostructures 10, 10′, 11, 11′ in the liquid carrier ranges from about 50 million nanostructures 10, 10′, 11, 11′ per mL of liquid carrier to about 500 million nanostructures 10, 10′, 11, 11′ per mL of liquid carrier. This example concentration may be increased if the size of the nanostructures 10, 10′, 11, 11′ remains the same and the density of the depressions 22 is increased.

Surfactants/dispersants, such as sodium dodecyl sulfate (e.g., sodium dodecyl sulfate (SDS), cetyltrimethylammonium bromide (CTAB)) may also be included in the liquid carrier. This suspension may be used for off-flow cell template strand preparation and amplification (e.g., when the functionalized nanostructure 10′ or 11′ is used), and then may be incorporated into the flow cell 20, 20′ for sequencing. Alternatively, the suspension may be used for on-flow cell template strand preparation and amplification (e.g., when the functionalized nanostructure 10 or 11 is used).

The liquid carrier may also include a metal chloride salt, such as sodium chloride or potassium chloride.

In a specific example, the liquid carrier includes a buffer (e.g., a phosphate buffer), a metal chloride salt, formamide, a surfactant, and water.

### Mechanical Loading Beads

The kit for biological sequencing operations disclosed herein further includes the plurality of mechanical loading beads 32 (shown in FIG. 3B).

Each of the plurality of mechanical loading beads 32 has a diameter (referred to herein as a “third diameter” D₃, see FIG. 3B) that is greater than the first diameter D₁ of each of the depressions 22. Because the second diameter D₂ (of each of the plurality of functionalized nanostructures 10, 10′, 11, 11′) is less than or equal to the first diameter D₁ (of each of the plurality of depressions 22), it is to be understood that the third diameter D₃ is also greater than the second diameter D₂ of the nanostructures 10, 10′, 11, 11′. The third diameter D₃ is at least one order of magnitude greater than the first diameter D₁. In an example, the third diameter D₃ ranges from about 100 μm to about 500 μm. In further examples, the third diameter D₃ ranges from about 125 μm to about 250 μm, or from about 150 μm to about 350 μm.

In an example, each of the plurality of mechanical loading beads 32 includes a material that is selected from the group consisting of a metal, a metal alloy, a polymer glass, or a combination thereof. In one specific example, each of the plurality of mechanical loading beads 32 includes stainless steel. In yet another specific example, each of the plurality of mechanical loading beads 32 consists of stainless steel. In yet another specific example, each of the plurality of mechanical loading beads 32 includes a material having a hardness similar to that of stainless steel.

The size of the mechanical loading beads 32 may also be expressed as a ratio (relative to the size of the plurality of functionalized nanostructures 10, 10′, 11, 11′). As an example, a size ratio of each of the mechanical loading beads 32 to each of the plurality of functionalized nanostructures 10, 10′, 11, 11′ is about 2:1.

The mechanical loading beads 32 may be suspended in a liquid carrier. The liquid carrier may be any of the example liquid carriers set forth herein in regard to the carrier for the functionalized nanostructures 10, 10′, 11, 11′.

### Method for Making the Non-Pre-Clustered Functionalized Nanostructure 10, 11

To make the functionalized nanostructure 10 shown in FIG. 1A, the hydrogel material is coated on the nanostructure core 12 to form the coating 14. The hydrogel material may be coated on the nanostructure core 12 using any suitable deposition technique. Examples of suitable deposition techniques include dip coating, dunk coating, spin coating, spray coating, puddle dispensing, ultrasonic spray coating, etc. In an example, the nanostructure core 12 may be suspended in the polymeric hydrogel material and exposed to conditions (e.g., heat) that will initiate the attachment of the polymeric hydrogel to the nanostructure core 12 for forming the coating 14. The type of attachment that is formed will depend upon the chemistry of the hydrogel material and the nanostructure core 12.

As described, in some examples, the nanostructure core 12 may include a silane that attaches the polymeric hydrogel material (e.g., of the hydrogel coating 14) to the nanostructure core 12.

Prior to forming the functionalized nanostructure 10, the hydrogel material may be prepared by polymerizing the monomer(s) that are to form the hydrogel coating 14. The polymerization process and process conditions will depend upon the monomer(s) included in the hydrogel material. In an example, the hydrogel material may be synthesized using reversible addition-fragmentation chain transfer (RAFT) polymerization. While RAFT polymerization may be used, it is to be understood that other polymerization processes may also be used. Other suitable polymerization processes include atom transfer radical polymerization (ATRP), nitroxide mediated radical (NMP) polymerization in combination with RAFT or ATRP, NMP with an additional cross-linking step, cobalt-mediated polymerization, group transfer polymerization (GTP), ring opening polymerization (ROP), ionic polymerization, or any other polymerization process that either directly or indirectly yields the desired linear or branched architecture.

Once the hydrogel material is formed and coated on the core 12 to form the hydrogel coating 14, the primers 8A, 8B may be grafted to the hydrogel coating 14. Grafting may involve dunk coating, which involves immersing the coated nanostructure core (12 with 14 thereon) in a primer solution or mixture, which may include the primer(s) 8A, 8B, water, a buffer, and a catalyst. Other grafting techniques may involve spray coating, puddle dispensing, or another suitable method that will attach the primer(s) 8A, 8B to the hydrogel coating 14. With any of the grafting methods, the primers 8A, 8B react with reactive groups of the hydrogel coating 14.

In other examples, the primers 8A, 8B are grafted to the hydrogel material before the hydrogel coating 14 is formed on the core 12. In this example, the core 12 may be suspended in the pre-grafted polymeric hydrogel material and exposed to conditions (e.g., heat) that will initiate the attachment of the pre-grafted polymeric hydrogel material to the core 12. In these examples, additional grafting is not performed.

To make the functionalized nanostructure 11 shown in FIG. 1A, the primers 8A, 8B are grafted to the hydrogel core 12′. The hydrogel core 12′ may be formed by emulsion polymerizing the monomer(s) in the presence of seed latexes and a surfactant, the latter of which promotes the coagulation of particles forming the nanostructures. Particle growth depends on the nucleation speed, and can be controlled by adjusting the monomer ratio, the conversion rate, the polymerization temperature, etc. Any of the grafting techniques disclosed herein may be used to attach the primers 8A, 8B to the hydrogel core 12′.

These functionalized nanostructures 10, 11 may be used in an on-flow cell amplification process for the generation of template nucleic acid strands 18.

### Method for Making the Pre-Clustered Functionalized Nanostructure 10′, 11′

The functionalized nanostructures 10, 11 may be used in an off-flow cell amplification process for the generation of template nucleic acid strands 18 (FIG. 1B) that are attached to the hydrogel coating 14 or hydrogel core 12′. This forms the pre-clustered nanostructures 10′, 11′, which can then be used in sequencing.

At the outset of template strand formation, library templates may be prepared from any nucleic acid sample (e.g., a DNA sample or an RNA sample). The DNA nucleic acid sample may be fragmented into single-stranded, similarly sized (e.g., <1000 bp) DNA fragments. The RNA nucleic acid sample may be used to synthesize complementary DNA (cDNA), and the cDNA may be fragmented into single-stranded, similarly sized (e.g., <1000 bp) cDNA fragments. During preparation, adapters may be added to the ends of any of the fragments. Through reduced cycle amplification, different motifs may be introduced in the adapters, such as sequencing primer binding sites, indices, and regions that are complementary to the primers 8A, 8B on the functionalized nanostructures 10, 11. In some examples, the fragments from a single nucleic acid sample have the same adapters added thereto. The final library templates include the DNA or cDNA fragment and adapters at both ends. The DNA or cDNA fragment represents the portion of the final library template that is to be sequenced.

A plurality of library templates may be introduced to a suspension containing the functionalized nanostructures 10, 11, where the suspension further includes the liquid carrier. Within the suspension, multiple library templates are individually hybridized, for example, to one of two types of primers 8A, 8B, which are immobilized to the functionalized nanostructures 10, 11. In some examples, one library template is hybridized to one functionalized nanostructure 10, 11. In other examples, multiple library templates are hybridized to one functionalized nanostructure 10, 11.

Amplification of the template nucleic acid strand(s) on the functionalized nanostructures 10, 11 may be initiated to form a cluster of the template strands 18 across the nanostructure surface. This generates pre-clustered nanostructures 10′, 11′. In one example, amplification involves cluster generation. In one example of cluster generation, the library templates are copied from the hybridized primers by 3′ extension using a high-fidelity DNA polymerase. The original library templates are denatured, leaving the copies immobilized all around the functionalized nanostructures 10, 11. Isothermal bridge amplification or some other form of amplification may be used to amplify the immobilized copies. For example, the copied templates loop over to hybridize to an adjacent, complementary primer 8A or 8B, and a polymerase copies the copied templates to form double stranded bridges, which are denatured to form two single stranded strands. These two strands loop over and hybridize to adjacent, complementary primers 8A or 8B and are extended again to form two new double stranded loops. The process is repeated on each template copy by cycles of isothermal denaturation and amplification to create dense clonal clusters on the functionalized nanostructures 10, 11. Each cluster of double stranded bridges is denatured. In an example, the reverse strand is removed by cleaving at the cleavage site (e.g., specific base cleavage), leaving forward template strands. In another example, the forward strand is removed by cleaving at the cleavage site, leaving reverse template strands. Clustering results in the formation of the pre-clustered nanostructures 10′, 11′, which includes several template strands 18 immobilized on the functionalized nanostructures 10, 11. This example of clustering is referred to as bridge amplification, and is one example of the amplification that may be performed. It is to be understood that other amplification techniques may be used, e.g., exclusion amplification.

When a single library template is hybridized and amplified on a single functionalized nanostructure 10, 11, the resulting pre-clustered nanostructure 10′, 11′ includes a monoclonal cluster of template strands 18.

The pre-clustered nanostructures 10′, 11′ may be washed to remove unreacted library templates, etc. and suspended in a fresh carrier liquid.

### Method of Using the Flow Cell

An example of a method of using the flow cell 20 is depicted in FIG. 3A through FIG. 3C. The example shown in these figures includes introducing the suspension including the liquid carrier and the plurality of functionalized nanostructures 10, 10′, 11, or 11′ into the flow cell 20 including the plurality of depressions 22 having the first diameter D₁, wherein each of the plurality of functionalized nanostructures 10, 10′, 11, or 11′ has the second diameter D₂ that is equal to or less than the first diameter D₁, and each of the plurality of functionalized nanostructures 10, 10′, 11, or 11′ includes: the nanostructure core 12, 12′; and a plurality of primers 8A, 8B attached to the nanostructure core 12, 12′ (shown in FIG. 3A); while the suspension is in contact with the flow cell 20, introducing a plurality of mechanical loading beads 32 having a third diameter Ds that is greater than the first diameter D₁, thereby forcing at least some of the plurality of functionalized nanostructures 10, 10′, 11, or 11′ into at least some of the plurality of depressions 22 (shown in FIG. 3B); and removing, from the flow cell 20, the plurality of mechanical loading beads 32 and at least some other of the plurality of functionalized nanostructures 10, 10′, 11, or 11′ that are not forced into the at least some of the plurality of depressions 22 (shown in FIG. 3C).

As shown in FIG. 3A, this example method includes introducing the suspension including the plurality of functionalized nanostructures 10, 10′, 11, or 11′ into the flow cell 20. The flow cell 20 shown in FIG. 3A includes two patterned structures 24A and 24B bonded together at the bonding region 23 via the spacer layer 34. It is to be understood, however, that the flow cell 20 may include a single patterned structure 24A bonded to the lid (not shown), or a single patterned structure 24A that is an open-wafer substrate (e.g., a single substrate 24A without a bonded lid or second substrate 24B bonded thereto).

The introduction of the plurality of functionalized nanostructures 10, 10′, 11, or 11′ may be performed via the flow cell inlet described herein (not shown).

In some examples of the method, prior to introducing the suspension into the flow cell 20, the method further comprises introducing a template DNA strand to the suspension, wherein the template DNA strand becomes attached to the at least one of the plurality of primers 8A, 8B. The suspension containing the template DNA strand is exposed to conditions that initiate amplification as described herein which forms the amplicons/DNA template strands 18. In these examples, the pre-clustered functionalized nanostructure 10′ or 11′ is formed and is introduced into the flow channel 21 of the flow cell 20.

As shown in FIG. 3B, the method proceeds by introducing the plurality of mechanical loading beads 32 into the flow cell 20. The plurality of mechanical loading beads 32 may include any suitable material disclosed herein. The introduction of the mechanical loading beads 32 into the flow cell 20 displaces the plurality of functionalized nanostructures 10, 10′, 11, or 11′, such that individual functionalized nanostructures 10, 10′, 11, or 11′ become loaded into the plurality of depressions 22. In other words, the mechanical loading beads 32 interact with the functionalized nanostructures 10, 10′, 11, or 11′ and force at least some of them into at least some of the depressions 22.

The mechanical loading beads 32 may be loaded into the flow cell 20 in an amount of about 1.5 g of beads 32 per 2125 mm² of flow cell surface area to about 2.5 g of beads 32 per 2125 mm² of flow cell surface area. In a specific example, the mechanical loading beads 32 may be present in the flow cell 20 in an amount of about 1.8 g of beads 32 per 2125 mm² of flow cell surface area.

In some examples, the fixture used to introduce the plurality of mechanical loading beads 32 into the flow cell 20 may be set to repeatedly load the same amount of mechanical loading beads 32 a predetermined number of times after the prior loading has been removed. In one example, the mechanical loading beads 32 are introduced twice.

In other examples, after the mechanical loading beads 32 are used to introduce the functionalized nanostructures 10, 10′, 11, 11′ into the flow cell 20, shaking is performed to further facilitate the loading of the nanostructures 10, 10′, 11, 11′ into the depressions 22. Shaking may be performed using any suitable instrument, such as an agitation apparatus operating at 200 rpm to 400 rpm.

As described, each of the plurality of mechanical loading beads 32 has the third diameter D₃ that is greater than the first diameter D₁ (e.g., of each of the plurality of depressions 22). Further, each of the plurality of functionalized nanostructures 10, 10′, 11, or 11′ has the second diameter D₂ that is less than or equal to the first diameter D₁ (e.g., of each of the plurality of depressions 22). As such, each of the plurality of functionalized nanostructures 10, 10′, 11, or 11′ is capable of becoming loaded into the depressions 22 by the plurality of mechanical loading beads 32 (due to the smaller second diameter D₂ of the nanostructure 10, 10′, 11, or 11′ relative to the first diameter D₁ of the depressions 22). Further, the mechanical loading beads 32 remain outside of the depressions 22 during the loading process (due in part to the larger third diameter D₃ of the loading beads 32 relative to the first diameter D₁ of the depressions 22).

In an example, the first diameter D₁ ranges from about 250 nm to about 1000 nm. In an example, the second diameter D₂ ranges from about 100 nm to about 1000 nm. In an example, the third diameter D₃ ranges from about 100 μm to about 500 μm.

Depending on the diameters D₁ and D₂ that are used, in some examples, multiple functionalized nanostructures 10, 10′, 11, or 11′ are loaded into a single depression 22 (as shown in one of the depressions 22 of FIG. 2B). In one example, anywhere from one functionalized nanostructure 10, 10′, 11, or 11′ to six functionalized nanostructures 10, 10′, 11, or 11′ may be introduced into a single depression 22.

As shown in FIG. 3C, the method then proceeds with removing the plurality of mechanical loading beads 32 from the flow cell 20 (e.g., via the outlet of the flow cell 20 described herein, outlet not shown in the figure). During the removal process, at least some of the plurality of functionalized nanostructures 10, 10′, 11, or 11′ that do not become deposited/loaded into the depressions 22 may also be removed. Removal of the unloaded nanostructures 10, 10′, 11, 11′ and the mechanical loading beads 32 may be accomplished by flushing the flow cell 20 with a suitable aqueous solution (e.g., water), or by manually removing the unloaded nanostructures 10, 10′, 11, 11′ and the beads 32 via wiping. It is believed that the loaded nanostructures 10, 10′, 11, 11′ remain within the depressions 22, due, in part, to Van der Waals interactions between the hydrogel coating 14 or hydrogel core 12′ of the functionalized nanostructure 10, 10′, 11, 11′ and the substrate 16 or layer 28 that forms the depressions 22. If the nanostructure core 12 is not a hydrogel and does not include the hydrogel coating 14, the size of the nanostructures and the size of the depressions 22 may be matched in order to retain the nanostructures in the depressions 22.

In some examples, after removing the mechanical loading beads 32 from the flow cell 20, the method further comprises introducing a template DNA strand 18 into at least one of the plurality of depressions 22, wherein the template DNA strand 18 becomes attached to the at least one of the plurality of primers 8A, 8B. In these examples, the non-pre-clustered functionalized nanostructures 10 or 11 are introduced into the flow cell 20 (at the step shown in FIG. 3A).

### Sequencing Method

The pre-clustered or non-preclustered nanostructures 10, 10′, 11, or 11′ may be used in sequencing on the flow cell 20.

When the pre-clustered nanostructures 10′ or 11′ are used, the suspension including the pre-clustered nanostructures 10′, 11′ (which includes a cluster of the template strands 18) may be introduced into the flow cell 20 including the plurality of depressions 22. The pre-clustered nanostructures 10′, 11′ are loaded into the depressions 22 using the mechanical loading beads 32.

Sequencing primers may then be introduced to the flow cell 20. The sequencing primers hybridize to a complementary portion of the sequence of the template strands 18 that are attached to the pre-clustered nanostructures 10′, 11′ (which are within the depressions 22 of the flow cell 20). The sequencing primers render the template strands 18 ready for sequencing.

An incorporation mix including labeled nucleotides may then be introduced into the flow cell 20, e.g., via the inlet. In addition to the labeled nucleotides, the incorporation mix may include water, a buffer, and polymerases capable of nucleotide incorporation. When the incorporation mix is introduced into the flow cell 20, the mix enters the flow channel 21, and contacts the sequence ready pre-clustered nanostructures 10′, 11′.

The incorporation mix is allowed to incubate in the flow cell 20, and labeled nucleotides (including optical labels) are incorporated by respective polymerases into the nascent strands along the template strands 18 on each of the pre-clustered nanostructures 10′, 11′. During incorporation, one of the labeled nucleotides is incorporated, by a respective polymerase, into one nascent strand that extends one sequencing primer and that is complementary to one of the template strands 18. Incorporation is performed in a template strand dependent fashion, and thus detection of the order and type of labeled nucleotides added to the nascent strand can be used to determine the sequence of the template strand 18. Incorporation occurs in at least some of the template strands 18 across the pre-clustered nanostructures 10′, 11′ during a single sequencing cycle.

The incorporated labeled nucleotides may include a reversible termination property due to the presence of a 3′ OH blocking group, which terminates further sequencing primer extension once the labeled nucleotide has been added. After a desired time for incubation and incorporation, the incorporation mix, including non-incorporated labeled nucleotides, may be removed from the flow cell 20 during a wash cycle. The wash cycle may involve a flow-through technique, where a washing solution (e.g., buffer) is directed into, through, and then out of flow channel 21, e.g., by a pump or other suitable mechanism.

Without further incorporation taking place, the most recently incorporated labeled nucleotides can be detected through an imaging event. During the imaging event, an illumination system may provide an excitation light to the flow cell 20. The optical labels of the incorporated labeled nucleotides emit optical signals in response to the excitation light.

After imaging is performed, a cleavage mix may then be introduced into the flow cell 20. In an example, the cleavage mix is capable of i) removing the 3′ OH blocking group from the incorporated nucleotides, and ii) cleaving the optical label from the incorporated nucleotide. Examples of 3′ OH blocking groups and suitable de-blocking agents/components in the cleavage mix may include: ester moieties that can be removed by base hydrolysis; allyl-moieties that can be removed with NaI, chlorotrimethylsilane and Na₂S₂O₃ or with Hg(II) in acetone/water; azidomethyl which can be cleaved with phosphines, such as tris(2-carboxyethyl) phosphine (TCEP) or tri (hydroxypropyl)phosphine (THP); acetals, such as tert-butoxy-ethoxy which can be cleaved with acidic conditions; MOM (—CH₂OCH₃) moieties that can be cleaved with LiBF₄ and CH₃CN/H₂O; 2,4-dinitrobenzene sulfenyl which can be cleaved with nucleophiles such as thiophenol and thiosulfate; tetrahydrofuranyl ether which can be cleaved with Ag(I) or Hg(II); and 3′ phosphate which can be cleaved by phosphatase enzymes (e.g., polynucleotide kinase). Examples of suitable optical label cleaving agents/components in the cleavage mix may include: sodium periodate, which can cleave a vicinal diol; phosphines, such as tris(2-carboxyethyl) phosphine (TCEP) or tris(hydroxypropyl)phosphine (THP), which can cleave azidomethyl linkages; palladium and THP, which can cleave an allyl; bases, which can cleave ester moieties; or any other suitable cleaving agent.

Additional sequencing cycles may then be performed until the template strands 18 are sequenced.

When the non-pre-clustered nanostructures 10 or 11 are used, the suspension including the non-pre-clustered nanostructures 10, 11 may be introduced into the flow cell 20 including the plurality of depressions 22. The non-pre-clustered nanostructures 10, 11 are and loaded into the depressions 22 using the mechanical loading beads 32.

In this example, amplification is performed on the flow cell 20. To initiate amplification, a plurality of library templates may then be introduced into the flow cell 20, whereupon the library templates become individually attached to primers 8A, 8B of the functionalized nanostructures 10, 11 that are in the depressions 22. Amplification of the library templates generates the amplicons/DNA template strands 18. A wash cycle may be performed to remove unreacted library templates.

Sequencing primers may then be introduced to the flow cell 20, which now includes clustered nanostructures (similar to structures 10′, 11′). The sequencing primers hybridize to a complementary portion of the sequence of the template strands 18 that are attached to the clustered nanostructures (which are within the depressions 22 of the flow cell 20). The sequencing primers render the template strands 18 ready for sequencing.

An incorporation mix including labeled nucleotides may then be introduced into the flow cell 20, e.g., via the inlet. In addition to the labeled nucleotides, the incorporation mix may include water, a buffer, and polymerases capable of nucleotide incorporation. When the incorporation mix is introduced into the flow cell 20, the mix enters the flow channel 21, and contacts the sequence ready clustered nanostructures.

The incorporation mix is allowed to incubate in the flow cell 20, and labeled nucleotides (including optical labels) are incorporated by respective polymerases into the nascent strands along the template strands 18 on each of the clustered nanostructures. During incorporation, one of the labeled nucleotides is incorporated, by a respective polymerase, into one nascent strand that extends one sequencing primer and that is complementary to one of the template strands 18. Incorporation is performed in a template strand dependent fashion, and thus detection of the order and type of labeled nucleotides added to the nascent strand can be used to determine the sequence of the template strand 18. Incorporation occurs in at least some of the template strands 18 across the clustered nanostructures during a single sequencing cycle.

The incorporated labeled nucleotides may include a reversible termination property due to the presence of a 3′ OH blocking group, which terminates further sequencing primer extension once the labeled nucleotide has been added. After a desired time for incubation and incorporation, the incorporation mix, including non-incorporated labeled nucleotides, may be removed from the flow cell 20 during a wash cycle. The wash cycle may involve a flow-through technique, where a washing solution (e.g., buffer) is directed into, through, and then out of flow channel 21, e.g., by a pump or other suitable mechanism.

Without further incorporation taking place, the most recently incorporated labeled nucleotides can be detected through an imaging event. During the imaging event, an illumination system may provide an excitation light to the flow cell 20. The optical labels of the incorporated labeled nucleotides emit optical signals in response to the excitation light.

After imaging is performed, a cleavage mix may then be introduced into the flow cell 20. In an example, the cleavage mix is capable of i) removing the 3′ OH blocking group from the incorporated nucleotides, and ii) cleaving the optical label from the incorporated nucleotide. Examples of 3′ OH blocking groups and suitable de-blocking agents/components in the cleavage mix may include any of those described herein.

Additional sequencing cycles may then be performed until the template strands 18 are sequenced.

To further illustrate the present disclosure, an example is given herein. It is to be understood that this example is provided for illustrative purposes and is not to be construed as limiting the scope of the present disclosure.

### Non-Limiting Working Example

This example was performed to compare sequencing metrics obtained from mechanically loaded non-pre-clustered functionalized nanostructures that were exposed to on-flow cell library preparation with those obtained from a commercially available patterned flow cell, namely a HISEQX™ flow cell from Illumina Inc. The commercially available flow cell includes the polymeric hydrogel (PAZAM) and grafted primers (P5 and P7) within each of the depressions.

An example of an open-wafer patterned flow cell (referred to as “first flow cell”) was used for loading non-pre-clustered functionalized nanostructures. The first flow cell included depressions imprinted into a resin layer. The depressions of the first flow cell were about 360 nm in diameter and did not include any polymeric hydrogel or primers therein (the first flow cell is similar to one patterned substrate of the HISEQX™ flow cell without any polymeric hydrogel or primers). Non-pre-clustered functionalized nanostructures were prepared using 300 nm silica particles, a PAZAM polymeric hydrogel coating, and P5 and P7 primers grafted to the hydrogel coating. The non-pre-clustered nanostructures were introduced into the flow channel and the depressions of the first flow cell using a plurality of stainless steel mechanical loading beads (sized 300 nm) in accordance with the method described herein. The loading beads were suspended in a buffer solution prior to being used to introduce the nanostructures into the depressions, and the bead suspension was applied over the non-pre-clustered nanostructures in the flow channel. The mechanical loading beads (and the functionalized nanostructures that remained outside of the depressions) were removed from interstitial regions of the flow cell using an ethanol wipe. Each of four different concentrations of PhiX genome library fragments (10 pM, 20 pM, 30 pM, and 40 pM) was respectively introduced into four lanes of the first flow cell and all of the fragments were amplified on board the cell flow cell using a cBOT™ instrument (Illumina Inc.) using the TRUSEQ™ reagents. Paired end sequencing (300 cycles) was performed on HISEQX™ sequencer.

As mentioned, for comparison, the commercially available patterned flow cell was used and is referred to in this example as the “second flow cell.” Library fragments of the PhiX genome were introduced into the second flow cell and amplified using EXAMP™ reagents (Illumina Inc.). 40 PM of Phix genome library fragments was introduced into one lane of the second flow cell and was amplified on board the cell flow cell using a cBOT™ instrument (Illumina Inc.) using the TRUSEQ™ reagents. Paired end sequencing (300 cycles) was performed on HISEQX™ sequencer.

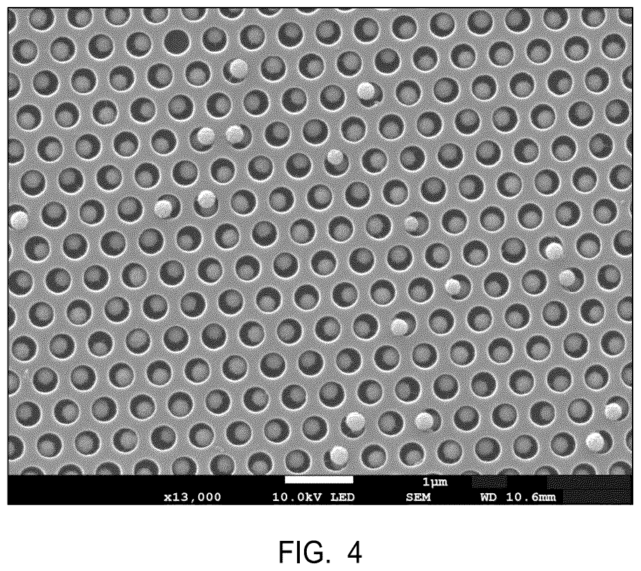

After 72 cycles of sequencing, a portion of the first flow cell was imaged using scanning electronic microscopy (SEM). This image is shown in FIG. 4. As depicted almost all of the depressions contained one or two clustered functionalized nanostructures.

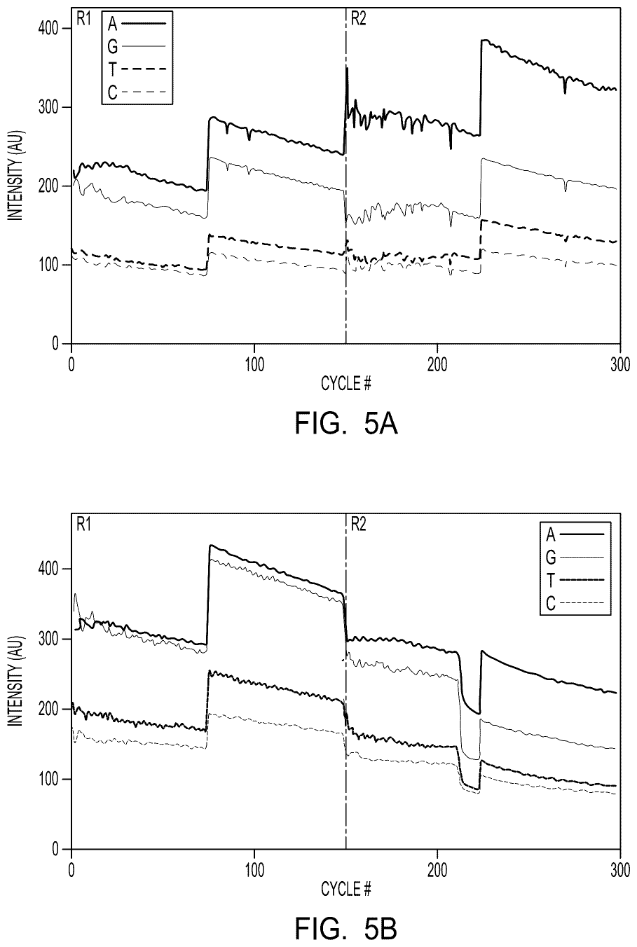

FIG. 5A and FIG. 5B depict an average intensity trace for all lanes in all four color channels for the first and second flow cells, respectively. As is demonstrated by the figures, the first flow cell including the on-board functionalized nanostructures displayed a high degree of fluorescence (in terms of intensity values) during the sequencing run and was comparable to the commercially available second flow cell.

Table 1 depicts sequencing metrics for a tile of each one of the lanes of the first flow cell containing the different concentration of library fragments, and for a tile of one of the lanes of the second flow cell. A tile is an area of a swath of a flow cell lane that is analyzed separately from other tiles. The sequencing metrics include % passing filter (% PF), % Occupancy, % Aligned, and P90.

Passing filter (PF) is the metric used to describe clusters which pass a chastity threshold and are used for further processing and analysis of sequencing data. The % PF calculation involves the application of a chastity filter to each cluster. “Chastity” is defined as the ratio of the brightest base intensity divided by the sum of the brightest and second brightest base intensities. Clusters “pass filter” if no more than 1 base call has a chastity value below 0.6 in the first 25 cycles. This filtration process removes the least reliable clusters from the image analysis results. As such, a higher % passing filter (% PF) result indicates an increased yield of usable sequencing data.

% Occupancy is a quantitative measurement of the percentage of depressions that are occupied by a cluster of amplicons (i.e., the percentage of depressions from which a fluorescence signal is detected, and thus by extension, containing a cluster).

% Aligned is the percentage of reads that are aligned to the reference genome. A higher mapped read (or aligned) percentage is indicative of the accuracy of the sequencing.

P90 shows the intensity by color of the 90% percentile of the data for each cycle.

TABLE 1

| | % | % | % | |
|---|---|---|---|---|
| | PF | Occupancy | Aligned | P90 |
| First flow cell tile - 10 pM PhiX | 35.8 | 43.7 | 89.2 | 183 |
| First flow cell tile - 20 pM PhiX | 44.9 | 58.1 | 98.3 | 240 |
| First flow cell tile - 30 pM PhiX | 53.3 | 68.1 | 99.4 | 292 |
| First flow cell tile - 40 pM PhiX | 49.2 | 69.8 | 99.1 | 305 |
| Second flow cell tile - 40 pM PhiX | 48.6 | 51.5 | 99.6 | 425 |

The data in Table 1 illustrates that the clustered functionalized nanostructures are as effective for sequencing as the commercially available flow cell.

### Additional Notes

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such values or sub-ranges were explicitly recited. For example, a range from about 2 mm to about 300 mm, should be interpreted to include not only the explicitly recited limits of from about 2 mm to about 300 mm, but also to include individual values, such as about 40 mm, about 250.5 mm, etc., and sub-ranges, such as from about 25 mm to about 175 mm, etc.

Furthermore, when “about” and/or “substantially” are/is utilized to describe a value, they are meant to encompass minor variations (up to +/−10%) from the stated value.

While several examples have been described in detail, it is to be understood that the disclosed examples may be modified. Therefore, the foregoing description is to be considered non-limiting.

What is claimed is:

1. A kit for biological sequencing operations, comprising:
a flow cell including a plurality of depressions having a first diameter;
a suspension including:
a liquid carrier; and
a plurality of functionalized nanostructures dispersed throughout the liquid carrier, wherein each of the plurality of functionalized nanostructures has a second diameter that is equal to or less than the first diameter, and each of the plurality of functionalized nanostructures includes:
a nanostructure core; and
a plurality of primers attached to the nanostructure core; and
a plurality of mechanical loading beads having a third diameter that is greater than the first diameter.

2. The kit as defined in claim 1, wherein each of the plurality of mechanical loading beads comprises a material that is selected from the group consisting of a metal, a polymer, glass, or combination thereof.

3. The kit as defined in claim 1, wherein:
each of the plurality of functionalized nanostructures further includes a polymeric hydrogel coating attached to the nanostructure core;
each of the plurality of primers is attached to a side chain or arm of the polymeric hydrogel coating; and
each of the plurality of primers is functionalized with an azide group or an alkyne group.

4. The kit as defined in claim 1, wherein each of the plurality of depressions is separated from each other of the plurality of depressions by interstitial regions.

5. The kit as defined in claim 1, wherein each of the plurality of functionalized nanostructures further includes:
silane at a surface of the nanostructure core; and
a polymeric hydrogel coating attached to the silane.

6. The kit as defined in claim 1, wherein the first diameter ranges from about 250 nm to about 1000 nm.

7. The kit as defined in claim 1, wherein the second diameter ranges from about 100 nm to about 1000 nm.

8. The kit as defined in claim 1, wherein the third diameter ranges from about 100 μm to about 500 μm.

9. The kit as defined in claim 1, wherein the liquid carrier is an aqueous solution including a buffer, a polar aprotic solvent, or combinations thereof.

10. A method, comprising:
introducing a suspension including a liquid carrier and a plurality of functionalized nanostructures into a flow cell including a plurality of depressions having a first diameter, wherein each of the plurality of functionalized nanostructures has a second diameter that is equal to or less than the first diameter, and each of the plurality of functionalized nanostructures includes:
a nanostructure core; and
a plurality of primers attached to the nanostructure core;
while the suspension is in contact with the flow cell, introducing a plurality of mechanical loading beads having a third diameter that is greater than the first diameter, thereby forcing at least some of the plurality of functionalized nanostructures into at least some of the plurality of depressions; and
removing, from the flow cell, the plurality of mechanical loading beads and at least some other of the plurality of functionalized nanostructures that are not forced into the at least some of the plurality of depressions.

11. The method as defined in claim 10, wherein prior to introducing the suspension into the flow cell, the method further comprises introducing a template DNA strand to the suspension, wherein the template DNA strand becomes attached to the at least one of the plurality of primers.

12. The method as defined in claim 10, wherein after removing the mechanical loading beads from the flow cell, the method further comprises introducing a template DNA strand into at least one of the plurality of depressions, wherein the template DNA strand becomes attached to the at least one of the plurality of primers.

13. The method as defined in claim 10, further comprising preparing the plurality of functionalized nanostructures by:
applying a silane to the nanostructure core; and
attaching a polymeric hydrogel to the nanostructure core having the silane thereon.

14. The method as defined in claim 10, wherein the first diameter ranges from about 250 nm to about 1000 nm.

15. The method as defined in claim 10, wherein the second diameter ranges from about 100 nm to about 1000 nm.

16. The method as defined in claim 10, wherein the third diameter ranges from about 100 μm to about 500 μm.


SEQUENCE LISTING (13 sequences)

SEQ ID NO: 1 - DNA, 29 bp, Synthetic construct
  source 1..29: mol_type = other DNA
  modified_base 23: mod_base = OTHER; note = uracil
aatgatacggcgaccaccgagatctacac

SEQ ID NO: 2 - DNA, 29 bp, Synthetic construct
  source 1..29: mol_type = other DNA
  modified_base 23: mod_base = i
aatgatacggcgaccaccgaganctacac

SEQ ID NO: 3 - DNA, 29 bp, Synthetic construct
  source 1..29: mol_type = other DNA
  modified_base 23: mod_base = OTHER; note = alkene-thymidine
aatgatacggcgaccaccgaganctacac

SEQ ID NO: 4 - DNA, 24 bp, Synthetic construct
  source 1..24: mol_type = other DNA
  modified_base 22: mod_base = OTHER; note = 8-oxoguanine
caagcagaagacggcatacganat

SEQ ID NO: 5 - DNA, 24 bp, Synthetic construct
  source 1..24: mol_type = other DNA
  modified_base 20: mod_base = OTHER; note = 8-oxoguanine
caagcagaagacggcatacnagat

SEQ ID NO: 6 - DNA, 24 bp, Synthetic construct
  source 1..24: mol_type = other DNA
  modified_base 22: mod_base = OTHER; note = 8-oxoguanine
  modified_base 20: mod_base = OTHER; note = 8-oxoguanine
caagcagaagacggcatacnanat

SEQ ID NO: 7 - DNA, 24 bp, Synthetic construct
  source 1..24: mol_type = other DNA
  modified_base 22: mod_base = OTHER; note = uracil
caagcagaagacggcatacgatat

SEQ ID NO: 8 - DNA, 24 bp, Synthetic construct
  source 1..24: mol_type = other DNA
  modified_base 20: mod_base = OTHER; note = uracil
caagcagaagacggcatactagat

SEQ ID NO: 9 - DNA, 29 bp, Synthetic construct
  source 1..29: mol_type = other DNA
  modified_base 23: mod_base = OTHER; note = allyl-T
aatgatacggcgaccaccgaganctacac

SEQ ID NO: 10 - DNA, 35 bp, Synthetic construct
  source 1..35: mol_type = other DNA
gctggcacgtccgaacgcttcgttaatccgttgag

SEQ ID NO: 11 - DNA, 35 bp, Synthetic construct
  source 1..35: mol_type = other DNA
cgtcgtctgccatggcgcttcggtggatatgaact

SEQ ID NO: 12 - DNA, 35 bp, Synthetic construct
  source 1..35: mol_type = other DNA
acggccgctaatatcaacgcgtcgaatccgcaact

SEQ ID NO: 13 - DNA, 34 bp, Synthetic construct
  source 1..34: mol_type = other DNA
gccgcgttacgttagccggactattcgatgcagc